(19) United States
(12) Patent Application Publication
(10) Pub. No.: US 2011/0064723 A1
(43) Pub. Date: Mar. 17, 2011

(54) Formulation for room temperature stabilization of a live attenuated bacterial vaccine

(75) Inventors: Vu Truong-Le, Campbell, CA (US); Satoshi Ohtake, Milpitas, CA (US); Gary Chiueh, San Jose, CA (US); Russell A. Martin, Los Gatos, CA (US); Atul Saxena, Milpitas, CA (US); Binh V. Pham, Mountain View, CA (US); David Lechuga-Ballesteros, San Jose, CA (US)

(73) Assignee: Aridis Pharmaceuticals

(21) Appl. No.: 12/880,213

(22) Filed: Sep. 13, 2010

Related U.S. Application Data
(60) Provisional application No. 61/242,376, filed on Sep. 14, 2009.

(51) Int. Cl. A61K 39/395; A61K 39/00; A61K 39/12; A61K 39/02; A61K 39/085; A61K 39/09; A61K 39/112; A61K 39/108; A61K 39/04; A61K 39/104; A61K 39/07; A61P 37/04

(52) U.S. Cl. 424/130.1; 424/184.1; 424/204.1; 424/234.1; 424/243.1; 424/244.1; 424/258.1; 424/257.1; 424/248.1; 424/260.1; 424/246.1

(57) ABSTRACT

This invention provides methods and compositions for stabilizing proteins and vaccines in dried formulations. In particular, a cavitation method and compositions of preparing a dried vaccine are provided that stabilize the viability of live bacteria and live virus vaccines at room temperature.

### PRIORITY

This application claims priority from U.S. Provisional Ser. No. 61/242,376 filed Sep. 14, 2009. This U.S. Provisional application is incorporated herein by reference.

### STATEMENT CONCERNING FEDERALLY SPONSORED RESEARCH OR DEVELOPMENT

Certain aspects of the invention disclosed herein were made with United States government support under NIAID, NIH SBIR grant #1 R43 AI063829-01A1. The United States government may have certain rights in the invention.

### FIELD OF THE INVENTION

The present invention is in the field of preservation of biologic materials in storage. In particular, the invention is directed to methods and formulations for stabilizing live bacteria and live virus vaccines using a combination of constituents providing protection through the formation of a glassy matrix.

### BACKGROUND OF THE INVENTION

In comparison to inactivated or subunit-based vaccines, live attenuated bacterial vaccines have the advantage of mimicking the natural infection route of pathogens and thus effectively stimulating the desired mucosal immune responses. Furthermore, due to their ability to replicate in the host, live attenuated bacterial vaccines can usually promote long-lasting and strong immune responses (Ebensen, T., et al (2004) Novel Vaccine Strategies). In addition, possession of immune stimulatory macromolecules that act as adjuvants provides significant benefits to the use of live attenuated bacteria as vectors for delivering heterologous antigens (Loessner, H., et al (2008) Int. J. Med. Microbiol. 298, 21-26).

Despite these apparent advantages of live attenuated bacterial vaccines, very few bacterial-based vaccines have become commercially available (Levine, M. M., et al (1987) Lancet 1, 1049-1052). Safety issues are a major hurdle that slows down the development of such vaccines. Furthermore, maintaining the viability of live bacterial vaccines during long-term storage remains a challenge. With the development of modern molecular techniques, the safety of live attenuated bacterial vaccines has been greatly improved by the use of controlled mutagenesis to construct genetically defined and attenuated pathogens. Such mutations, however, can affect a range of metabolic and structural elements in bacterial cells and consequently often cause high sensitivity of attenuated bacteria to adverse environments such as UV radiation and elevated temperature (Corbel, M. J. (1996) Dev. Biol. Stand. 87, 113-124).

One of the few licensed live attenuated bacterial vaccines is the Ty21a typhoid vaccine. This is a gal E mutant developed from the wild type Salmonella typhi Ty2 strain (Germanier, R. and Furer, E. (1975) J Infect Dis 131, 553-558), which causes typhoid fever in humans. Typhoid fever is a life-threatening infection common in the developing world. In the United States, about 400 cases occur each year with at least 70% of the cases acquired while traveling internationally. Worldwide, there are about 21 million cases per year, with 200,000 deaths. Without therapy, the death rate ranges between 12 to 30% (Typhoid Fever, in Technical information, Centers for Disease Control and Prevention (2005)).

Two formulations for typhoid vaccine are commercially available. One is Ty21a, a live oral vaccine in the form of an enteric-coated capsule formulation. The second is a liquid formulation reconstituted from lyophilized organisms. Both formulations demonstrate excellent safety records with high efficacy and produce only a few minor adverse reactions (Black, R., et al (1983) Dev. Biol. Stand. 53, 9-14; Wandan, M. H., et al (1980) Bull World Health Organ 58, 469-474; Levine, M. M., et al (1987) Lancet 1, 1049-1052; Levine, M. M., et al (1999) Vaccine 17, Suppl 2, S22-27), although the liquid formulation is reported to be superior to the enteric-coated capsules (Levine, M. M., et al (1990) Lancet 336, 891-894). These vaccines, however, possess low thermal stability and lose viability when exposed to adverse conditions, such as UV radiation and elevated temperature (Corbel, M. J. (1996) Dev Biol Stand 87, 113-124). In the case of the lyophilized Ty21a vaccine, its shelf life is dependent on residual moisture, excipients, and processing temperatures during manufacturing (Cryz, S. J., Jr., et al (1996) Dev. Biol. Stand. 87, 277-281).

Compared to liquid formulations, solid formulations have multiple advantages such as avoidance of freeze-thaw stress, prevention of agitation/shear induced denaturation, and increased ease in shipping and distribution. Furthermore, solid formulations decrease molecular motions and water-involved degradation reactions; this results in improved stability and longer shelf-life of biopharmaceuticals.

Unlike freeze drying and spray drying, which expose the drug to low and high temperatures respectively, drying processes have been developed which can be conducted at room temperature. Annear described a drying process, involving foaming and cavitation, whereby Salmonella ndolo and Vibrio cholerae were dried under high vacuum, while the ampoules containing the bacterial suspensions were immersed in a water bath at 20° C. (Annear, D. I. (1958) Aust. J. Exp. Biol. Med. Sci. 36(3), 211-221). The suspensions were dried within 2 minutes and the drying was continued for at least 24 hours. Another process described by Annear further comprised of a secondary drying step, in which the dried vaccine (as described above) was immersed in a water bath at 100° C. and placed under high vacuum, during which the partly dried suspension expanded rapidly into a homogeneous white foam (Annear, D. I. (1970) J. Hyg. (Loud.) 68(3), 457-459). Stamp stabilized Chromobacterium prodigiosum by drying the bacterial suspension in a desiccator containing P₂O₅ at a pressure of 100-300 mm Hg for 2-3 days at room temperature (Stamp, L. (1947) J. Gen. Microbiology 1(2), 252-265). The bacterial titer obtained using this method was approximately 3 times higher compared to that obtained from lyophilization. For all of the published work described above, the actual sample temperature was not measured or controlled in a manner capable with the freeze dryer equipment technology available today. More recently, Bronshtein described a method to dry biologically active materials at a negative pressure sufficient to cause the solution to boil (Bronshtein, V., U.S. Pat. No. 5,766,520) while Roser described a method to dry biological macromolecules at a temperature above freezing in the presence of trehalose (Roser, B. J., U.S. Pat. No. 4,891,319). Both methods are similar to the process originally described in detail by Anner more than 40 years prior, in that the drying is conducted under non-freezing conditions, with the dehydration process being driven by the lowered hydrostatic pressure. The boiling process described by Bronshtein is a consequence of the decreased pressure, e.g. boiling point depression of the solvent, which results in expanded foam, as described by Annear (Annear, D. I. (1970) J. Hyg. (Loud.) 68(3), 457-459). Furthermore, samples dried according to Annear's process exhibited a wide range of temperature profiles during dehydration; depending on the drying temperature, the rate of pressure decrease, and the solution composition, some of the samples underwent freezing while others did not (Annear, D. I. (1961) Aust. J. Exp. Biol. Med. Sci. 39, 295-303). A similar method was described by Truong-Le, whereby the bioactive material, which was being dried at a temperature above freezing, was dried through the steps of freezing followed by sublimation (U.S. Pat. No. 7,135,180 issued to Truong-Le). The samples were frozen due to the low system pressure. The methods that followed Annear, namely those described by Stamp and Lord, appear to involve similar process ranges in pressure and temperature regimes, wherein the formulation would have undergone similar chemical-physical transitions. The present art involves unique formulations and a cavitation process wherein the process range (in temperature and pressure) and phase transitions, to which the formulations are being subjected through, are similar to a foam/cavitation process described by Annear, et al., but differs in its use of a pharmaceutical freeze drying equipment to afford better control of the process parameters than that originally described by Annear. Moreover, the pharmaceutical formulation disclosed herein is an improvement on that originally described by the works of Annear. The improved process control is designed to enhance consistency and reproducibility of the foam.

In view of the above, a need exists for a vaccine formulation, demonstrating improved stability, manufactured by a more controlled drying method. The present invention provides these and other features that will be apparent upon review of the following.

### SUMMARY OF THE INVENTION

The novel invention provides methods and compositions for stabilization of bacteria. In particular, the compositions employ a polyol, in combination with various other formulation constituents, to stabilize Salmonella in live oral vaccine formulations. Additionally, the current invention provides for methods to enhance the stability of live bacteria through pharmaceutical drying process and storage stability by affecting the growth and harvesting conditions of the bacteria. Methods of the invention include a description of processes employed to prepare a room-temperature stable dry vaccine.

In one aspect of vaccine compositions, the vaccine formulation includes a strain of Salmonella at a titer ranging from about 1×10⁸ to about 1×10¹⁰ cfu/mL, a polyol at a concentration ranging from about 5% to about 70% (w/v), a buffer ranging in concentration from about 5 mM to about 2M, an amino acid ranging in concentration from about 0.1% to about 5% (w/v), a plasticizer ranging in concentration from about 0.1% to about 5% by weight of said formulation, a polymer ranging in concentration from about 0.1% to about 20% (w/v), and a surfactant ranging in concentration from about 0.01% to about 1% by weight of said formulation. In preferred embodiments, the polyol is trehalose ranging in concentration between about 20% to about 40% (w/v), the buffer is potassium phosphate ranging in concentration from about 5 mM to about 100 mM, the amino acid is methionine ranging in concentration from about 0.1% to about 5% (w/v), the plasticizer is glycerol ranging in concentration from about 0.1% to about 5% by weight of said formulation, the polymer is gelatin ranging in concentration from about 0.1% to 10% (w/v), and the surfactant is block copolymers of polyethylene and polypropylene glycol ranging in concentration from about 0.01% to about 1% by weight of said formulation.

Particular constituents or proportions of constituents are identified herein as useful aspects of the compositions. For example, the polyol of the formulation can be sucrose, trehalose, sorbose, melezitose, sorbitol, stachyose, raffinose, fructose, mannose, maltose, lactose, arabinose, xylose, ribose, rhamnose, galactose, glucose, mannitol, xylitol, erythritol, threitol, sorbitol, glycerol, L-glyconate, dextrose, fucose, polyaspartic acid, inositol hexaphosphate (phytic acid), sialic acid, and N-acetylneuraminic acid-lactose, and/or the like. In other aspects of the invention, the pharmaceutically acceptable buffer can include potassium phosphate, sodium phosphate, sodium acetate, histidine, imidazole, sodium citrate, sodium succinate, ammonium bicarbonate, a carbonate, and/or the like. For enhanced stability of Salmonella, the preferred formulation pH can range from about pH 4.0 to about pH 10.0; more preferably from pH 6.0 to pH 8.0; and most preferably at about pH 7.0

In some cases, the formulation can usefully include a surfactant ranging from about 0.001% to about 2% by weight of said formulation. For example, the formulation can include polyethylene glycol, polypropylene glycol, polyethylene glycol/polypropylene glycol block copolymers, polyethylene glycol alkyl ethers, polyethylene glycol sorbitan monolaurate, polypropylene glycol alkyl ethers, polyethylene glycol/polypropylene glycol ether block copolymers, polyoxyethylenesorbitan monooleate, alkylarylsulfonates, phenylsulfonates, alkyl sulfates, alkyl sulfonates, alkyl ether sulfates, alkyl aryl ether sulfates, alkyl polyglycol ether phosphates, polyaryl phenyl ether phosphates, alkylsulfosuccinates, olefin sulfonates, paraffin sulfonates, petroleum sulfonates, taurides, sarcosides, fatty acids, alkylnaphthalenesulfonic acids, naphthalenesulfonic acids, lignosulfonic acids, condensates of sulfonated naphthalenes with formaldehyde and phenol, lignin-sulfite waste liquor, alkyl phosphates, quaternary ammonium compounds, amine, oxides, betaines, and/or the like. In preferred embodiments, the surfactant is present in the formulation at a concentration ranging from about 0.01% to about 1% by weight of said formulation.

In other embodiments, the formulation can further comprise of an amino acid selected from the group consisting of alanine, arginine, methionine, serine, lysine, histidine, glutamic acid, and/or the like. In preferred embodiments, the amino acid is present in the formulation at a concentration ranging from about 0.1% to about 5% (w/v). In another aspect of the invention, the formulation can further comprise of a plasticizer selected from the group consisting of glycerol, dimethylsulfoxide (DMSO), propylene glycol, ethylene glycol, oligomeric polyethylene glycol, sorbitol, and/or the like. In preferred embodiments, the plasticizer is present in the formulation at a concentration ranging from about 0.1% to about 5% by weight of said formulation.

In another embodiment, the formulation can usefully include from about 0.1% to about 20% (w/v) of a polymer. For example, the formulation can include gelatin, hydrolyzed gelatin, collagen, chondroitin sulfate, a sialated polysaccharide, water soluble polymers, polyvinyl pyrrolidone, actin, myosin, microtubules, dynein, kinetin, human serum albumin, and/or the like.

In another embodiment, the bacteria is grown in hypertonic media such as sodium chloride to condition the bacteria to be more resistant to osmotic stress experienced during water removal as part of the drying process.

In yet another embodiment, the bacteria is selected from the early stationary growth phase of the fermentation process.

In a most preferred embodiment, the bacteria is selected at the early stationary growth phase wherein the dry vaccine formulation comprises Salmonella at a titer ranging from about 1×10⁸ to about 1×10¹⁰ cfu/mL, trehalose ranging in concentration between about 20% to about 40% (w/v), potassium phosphate ranging in concentration from about 20 mM to about 40 mM, methionine ranging in concentration from about 0.5% to about 2% (w/v), glycerol ranging in concentration from about 0.5% to about 3% by weight of said formulation, gelatin ranging in concentration from about 2% to 6% (w/v), block copolymers of polyethylene and polypropylene glycol ranging in concentration from about 0.1% to about 0.5% by weight of said formulation, and with a formulation pH ranging from about pH 6.0 to about 8.0.

The present invention also includes methods to prepare room temperature stable dry vaccine-containing formulations. That is, the formulations of the current invention can be dried to form vaccines for storage and/or administration in non-liquid form. Methods of the current invention can provide a glassy pharmaceutical formulation by a cavitation method conducted using a freeze-dryer. In another aspect, the methods include the steps of lyophilizing the liquid formulation, e.g., to form a dry powder or cake. In yet another aspect, the liquid formulation can be spray dried to form powder particles or to form a dried layer on a surface.

The invention provides liquid or dry pharmaceutical formulation composition, comprising: at least one bioactive material in the form of a protein, a virus, or a bacteria; a polyol at a concentration ranging from about 10% to about 70% (w/v); a pharmaceutically acceptable buffer ranging from about 5 mM to about 100 mM; a plasticizer or a surfactant at a concentration ranging from about 0.1% to about 10% by weight of said formulation, or a plasticizer at about 0.1% to about 5% (w/v), or a plasticizer at 0.1% to 10% (w/v), or a plasticizer at 0.1% to 5% (w/v).

In another aspect, what is provided is the above composition, and related methods, wherein the bacteria is selected from the list consisting of Salmonella, Shigella, Listeria, Franciscella, E. coli, Pneumococcus, Mycobacterium, Pseudomonas, Staphylococcus, Streptococcus, and their mixtures thereof.

Moreover, what is provided is the above composition, and related method, wherein the virus is selected from the list consisting of rotavirus, adenovirus, measles virus, mumps virus, rubella virus, polio virus, influenza virus, parainfluenza virus, respiratory syncytial virus, herpes simplex virus, SARS virus, vaccinia, corona virus family members, cytomegalovirus, human metapneumovirus, filovirus, Epstein-Bar virus, and their mixtures thereof. What is encompassed are vaccines of recombinant bacteria, engineered to express antigens (see, e.g., WO2007117371 of Dubensky, et al.)

Additionally, what is contemplated is the above bacterial strains, and related methods,wherein the bacteria are recovered from the early stationary phase of bacterial fermentation growth, as well as the above bacteria, where the bacteria are grown in a hyperosmotic medium. What is also provided is the above growth medium, wherein the hyperosmotic medium contains NaCl ranging in concentration from about 10 mM to about 2M.

In another embodiment, what is provided is the above composition, and related methods, wherein a polyol is selected from the group consisting of sucrose, trehalose, sorbose, melezitose, sorbitol, stachyose, raffinose, fructose, mannose, maltose, lactose, arabinose, xylose, ribose, rhamnose, galactose, glucose, mannitol, xylitol, erythritol, threitol, sorbitol, glycerol, L-glyconate, dextrose, fucose, polyaspartic acid, inositol hexaphosphate (phytic acid), sialic acid, and N-acetylneuraminic acid-lactose.

In another aspect, the invention includes the above composition, wherein the polyol is sucrose present at a concentration ranging from about 10% to about 50% (w/v), and the above composition, wherein the polyol is trehalose present at a concentration ranging from about 10% to about 50% (w/v), and also the above composition, wherein a pharmaceutically acceptable buffer is selected from the group consisting of potassium phosphate, sodium phosphate, sodium acetate, histidine, imidazole, sodium citrate, sodium succinate, ammonium bicarbonate, a carbonate, and their mixtures thereof. Regarding buffers, what is provided is the above composition, and related methods, wherein the pharmaceutically acceptable buffer is potassium phosphate present at a concentration ranging from about 5 mM to about 100 mM. Regarding plasticizers, what is provided is the above composition, and related methods, wherein a plasticizer is selected from the group consisting of glycerol, dimethylsulfoxide (DMSO), propylene glycol, ethylene glycol, oligomeric polyethylene glycol, and sorbitol. Regarding surfactants, what is provided is the above composition, and related methods, wherein the surfactant is selected from a list of pharmaceutically acceptable surfactants which includes Tween 20, Tween 80, Span 20, and Pluronics poloxamer. Also, the invention provides the above matter, wherein the plasticizer is DMSO present at a concentration ranging from about 0.1% to about 10% by weight of said formulation, or wherein the plasticizer is present in a range of 0.1% to 10%, 0.2% to 10%, 1.0 to 10%, 4.0% to 10%, or 0.1% to 8%, 0.1% to 6%, 0.1% to 4%, or 0.1% to 2%, or any combination thereof.

Amino acid embodiments of the above compositions and methods are embraced, and these include pharmaceutical formulations that further comprises of an amino acid selected from the list that includes methione, glutamine, serine, arginine, lyisine, asperigine, leucine, glycine, and their mixtures thereof, as well as compositions wherein an amino acid is present at a concentration ranging from about 0.1% to about 5% (w/v), and also include compositions, wherein the amino acid is methionine present at a concentration ranging from about 0.1% to about 4% (w/v), and also encompass compositions, wherein the amino acid is arginine present at a concentration ranging from about 0.1% to about 4% (w/v).

Regarding polymers and buffers, what is provided are the above compositions and related methods, wherein the liquid or dry formulation composition further comprises of a polymer selected from the group consisting of gelatin, hydrolyzed gelatin, collagen, chondroitin sulfate, a sialated polysaccharide, water soluble polymers, polyvinyl pyrrolidone, actin, myosin, microtubules, dynein, kinetin, bovine serum albumin, or human serum albumin. Moreover, the polymer embodiments include the above, wherein a polymer is present at a concentration ranging from about 0.1% to about 20% (w/v), and the above embodiments, wherein the polymer is gelatin present at a concentration ranging from about 0.1% to about 10% (w/v), and also the above embodiments, wherein the buffer comprises a pH ranging from about pH 4 to about pH 10, and also the above embodiments, wherein the buffer comprises a pH ranging from about pH 6 to about pH 8.

What is also embraced by the present invention, is a dry vaccine composition of as described above, that is prepared by freeze drying, by spray drying.

Moreover, what is encompassed is a cavitation method conducted using a freeze-dryer to prepare glassy pharmaceutical formulations containing bioactive materials containing at least 10% solids by weight, wherein the formulation is subjected to a pressure of 100 mTorr to 50 Torr for 5 to 90 minutes, and this method wherein the freeze dryer chamber pressure is reduced gradually from ambient to 100 mTorr within 90 minutes, and this method, wherein the freeze dryer shelf temperature is set between −10° C. and 40° C.

In a multi-step embodiment of the present compositions and methods, what is provided is a method wherein the freeze dryer chamber pressure is reduced stepwise from ambient pressure to 50 Torr, to 4 Torr, to 1 Torr, and to 100 mTorr within 90 minutes. Use of a “freeze dryer” encompasses methods wherein the sample never freezes, and wherein the sample is substantially prevented from freezing.

What is also provided is the above method, wherein the process further comprises holding the reduced pressure and drying temperature for a time ranging from about 12 hours to about 5 days, and wherein the process encompasses the drying temperature raised to 25° C. or higher.

In various bioactive material embodiments, the bioactive material is selected from the group consisting of proteins, peptides, antibodies, enzymes, serums, vaccines, nucleic acids, adjuvants, liposomes, viruses, bacteria, prokaryotic cells, and eukaryotic cells, as well as bioactive material embodiments, wherein the bacteria is selected from the list consisting of Salmonella, Shigella, Listeria, Franciscella, E. coli, Pneumococcus, Mycobacterium, Pseudomonas, Staphylococcus, Streptococcus, and their mixtures thereof, and also bioactive material embodiments, wherein the virus is selected from the list consisting of rotavirus, adenovirus, measles virus, mumps virus, rubella virus, polio virus, influenza virus, parainfluenza virus, respiratory syncytial virus, herpes simplex virus, SARS virus, vaccinia, corona virus family members, cytomegalovirus, human metapneumovirus, filovirus, Epstein-Bar virus, and their mixtures thereof, and in yet another aspect, bioactive material embodiments, wherein the bioactive material is composed of a protein vaccine and one or more adjuvants.

The present invention, in further embodiments, encompasses the above methods, wherein the liquid or dry pharmaceutical formulation composition, comprising: at least one bioactive material in the form of a protein, a virus, or a bacteria; a polyol at a concentration ranging from about 10% to about 70% (w/v); a pharmaceutically acceptable buffer ranging from about 5 mM to about 100 mM; a plasticizer or a surfactant at a concentration ranging from about 0.1% to about 10% by weight of said formulation.

In bacterial embodiments of the above compositions and methods, what is encompassed is the above, wherein the bacteria is selected from the list consisting of Salmonella, Shigella, Listeria, Franciscella, E. coli, Pneumococcus, Mycobacterium, Pseudomonas, Staphylococcus, Streptococcus, and their mixtures thereof, and also the above, wherein the virus is selected from the list consisting of rotavirus, adenovirus, measles virus, mumps virus, rubella virus, polio virus, influenza virus, parainfluenza virus, respiratory syncytial virus, herpes simplex virus, SARS virus, vaccinia, corona virus family members, cytomegalovirus, human metapneumovirus, filovirus, Epstein-Bar virus, and their mixtures thereof.

In microbial culturing embodiments, what is provided is the above bacterial strains, wherein the bacteria are recovered from the early stationary phase of bacterial fermentation growth, or in another aspect wherein the bacteria are grown in a hyperosmotic medium, or in yet another aspect, the above growth medium, wherein the hyperosmotic medium contains NaCl ranging in concentration from about 10 mM to about 2M.

In polyol embodiments of the above compositions and methods, what is encompassed is the above, wherein a polyol is selected from the group consisting of sucrose, trehalose, sorbose, melezitose, sorbitol, stachyose, raffinose, fructose, mannose, maltose, lactose, arabinose, xylose, ribose, rhamnose, galactose, glucose, mannitol, xylitol, erythritol, threitol, sorbitol, glycerol, L-glyconate, dextrose, fucose, polyaspartic acid, inositol hexaphosphate (phytic acid), sialic acid, and N-acetylneuraminic acid-lactose, as well as the above compositions and methods, wherein the polyol is sucrose present at a concentration ranging from about 10% to about 50% (w/v), as well as the above compositions and methods, wherein the polyol is trehalose present at a concentration ranging from about 10% to about 50% (w/v).

In buffer embodiments of the above compositions and methods, what is embraced is the above, wherein a pharmaceutically acceptable buffer is selected from the group consisting of potassium phosphate, sodium phosphate, sodium acetate, histidine, imidazole, sodium citrate, sodium succinate, ammonium bicarbonate, a carbonate, and their mixtures thereof, and also the above, wherein the pharmaceutically acceptable buffer is potassium phosphate present at a concentration ranging from about 5 mM to about 100 mM.

In plasticizer embodiments, what is included is the above compositions and methods, wherein a plasticizer is selected from the group consisting of glycerol, dimethylsulfoxide (DMSO), propylene glycol, ethylene glycol, oligomeric polyethylene glycol, and sorbitol. Surfactant embodiments include the surfactant is selected from a list of pharmaceutically acceptable surfactants which includes Tween® 20, Tween® 80, Span® 20, and Pluronics® poloxamer. Plasticizer embodiments include DMSO present at a concentration ranging from about 0.1% to about 10% by weight of said formulation.

Amino acid embodiments include pharmaceutical formulations further comprising an amino acid selected from the list that includes methione, glutamine, serine, arginine, lyisine, asperigine, leucine, glycine, and their mixtures thereof, and also the above, wherein an amino acid is present at a concentration ranging from about 0.1% to about 5% (w/v), and also the above, wherein the amino acid is methionine present at a concentration ranging from about 0.1% to about 4% (w/v), and also the above, wherein the amino acid is arginine present at a concentration ranging from about 0.1% to about 4% (w/v).

Moreover, the above compositions and methods include the above, wherein the liquid or dry formulation composition further comprises of a polymer selected from the group consisting of gelatin, hydrolyzed gelatin, collagen, chondroitin sulfate, a sialated polysaccharide, water soluble polymers, polyvinyl pyrrolidone, actin, myosin, microtubules, dynein, kinetin, bovine serum albumin, or human serum albumin, as well as the above materials and methods, wherein a polymer is present at a concentration ranging from about 0.1% to about 20% (w/v), and also the above composition, wherein the polymer is gelatin present at a concentration ranging from about 0.1% to about 10% (w/v).

Without implying any limitation, still other embodiments of the present invention include the above compositions, and related methods, wherein the buffer comprises a pH ranging from about pH 4 to about pH 10, and also, wherein the buffer comprises a pH ranging from about pH 6 to about pH 8.

Moreover, what is provided is the above dry vaccine composition dry vaccine composition of claim prepared by a cavitation method, wherein a pharmaceutical formulation is subjected to a pressure of 100 mTorr to 50 Torr for 5 to 90 minutes using a freeze dryer, and yet in another aspect, what is provided is the above method, wherein the freeze dryer chamber pressure is reduced gradually from ambient to 100 mTorr within 90 minutes, and also what is provided is the above method, wherein the freeze dryer shelf temperature is set between −10° C. and 40° C. In still another aspect of the above compositions and methods, what is provided is the above method, wherein the freeze dryer chamber pressure is reduced stepwise from ambient pressure to 50 Torr, to 4 Torr, to 1 Torr, and to 100 mTorr within 90 minutes, and also, the above compositions and methods, wherein the process further comprises holding the reduced pressure and drying temperature for a time ranging from about 12 hours to about 5 days, and also the above compositions and methods, wherein the drying temperature is raised to 25° C. or higher.

In a preferred embodiments, what is provided is a cavitation-dried composition that comprises a biologically active sample, wherein the composition is prepared from a mixture of the biologically active sample and a formulation, wherein the mixture contains a polyol (20-70% w/v of mixture), and a plasticizer (0.1-10.0% w/w of mixture), at pH 6.0-8.5, wherein to prepare the composition, the mixture is subjected to a vacuum, wherein the mixture temperature, or shelf temperature, is maintained above the freezing point of the mixture, wherein a cavitation-dried structure is produced, and wherein the cavitation-dried structure is a foam or a film. Also, what is encompassed is the above cavitation-dried composition of, wherein the polyol is sucrose (20-50% w/v in mixture), trehalose (20-50% w/v in mixture), or combinations thereof. Moreover, what is embraced is the above cavitation-dried composition, wherein the plasticizer is glycerol, dimethylsulfoxide (DMSO), propylene glycol, ethylene glycol, oligomeric polyethylene glycol, or sorbitol. Additionally, what is contemplated is the above cavitation-dried composition, wherein the formulation contains an amino acid, where the concentration of the amino acid in the mixture is 0.5-5.0% w/v. Further, what is provided is the above cavitation-dried composition, wherein the formulation contains an amino acid, and wherein the amino acid is methionine, arginine, or combinations thereof, wherein the amino acid in the mixture is 0.5-5.0% w/v. Moreover, what is supplied is the above cavitation-dried composition, that contains a surfactant, wherein the concentration of the surfactant in the mixture is 0.01-5.0% w/v. In yet another aspect, what is contemplated is the above cavitation-dried composition, that contains a surfactant, and is a surfactant-containing composition, and wherein there is a ratio of residence of the biologically active sample at the surface of the surfactant-containing composition versus at the interior of the surfactant-containing composition, and wherein the surfactant results in a decrease in this ratio, as compared to the ratio in a second cavitation-dried composition that contains all of the components of the surfactant-containing composition but lacking the surfactant. In yet another embodiment, the invention includes the above cavitation-dried composition, that further comprises a surfactant, wherein the surfactant has the chemical composition of Tween20®, Span20®, Tween80®, or Pluronic® poloxamer. Moreover, the invention encompasses the above cavitation-dried composition, that has a specific surface area, and where the specific surface area is less than 0.3 meters squared per gram of mass. Additionally, what is embraces is the above cavitation-dried composition, that has a specific surface area, and where the specific surface area is less than 0.1 meters squared per gram of mass.

Film embodiments are also included. In yet another embodiment of the present invention, what is included is the above cavitation-dried composition that is a film.

Additionally, what is contemplated is the above, cavitation-dried composition wherein the formulation contains an amino acid and a plasticizer, wherein the amino acid reduces process loss, wherein the plasticizer reduces process loss, or wherein the combination of both amino acid and plasticizer in the formulation has an additive effect in reducing process loss. Further, what is included is the above cavitation-dried composition wherein the amino acid is methionine and the plasticizer is dimethylsulfoxide (DMSO). Also, what is encompassed is each of the above embodiments of the invention, wherein the formulation is from Table 1, 2, 5, or 8, as well as each of the embodiments, wherein the biologically active sample is bacteria or viruses.

In a microbial embodiment, what is provided is the above cavitation-dried composition, wherein the biologically active sample is bacteria, and wherein (a) the bacteria is harvested at stationary phase, (b) the bacterial growth medium is hyperosmotic, or (c) the bacteria is harvested at stationary phase and the bacterial growth medium is hyperosmotic, wherein process stability is increased in the hyperosmotic medium compared to process stability wherein the bacterial growth medium is iso-osmotic, and wherein process stability is increased when the bacteria are harvested in stationary phase compared to process stability when the bacteria are harvested in the log phase.

In immune-stimulatory and non-immune stimulatory embodiments, the present invention encompasses the above cavitation-dried composition, wherein the biologically active sample does not elicit an immune response against itself, or is engineered to prevent an immune response against itself; and also the above cavitation-dried composition, wherein the biologically active sample can elicit an immune response against itself.

In a methods embodiment, what is embraced is a method for preparing a cavitation-dried composition of a biologically active sample, from a mixture of a biologically active sample and a formulation, wherein the formulation comprises a polyol, and a plasticizer or a surfactant, and wherein the mixture is in a container, wherein the method comprises decreasing the chamber pressure in a stepwise manner to reduce the water content of the mixture, wherein the mixture temperature or the shelf temperature, is maintained above the freezing point of the mixture, wherein a foam or film is produced, and wherein the mixture does not freeze and the foam or film does not freeze.

In embodiments that relate to the time of steps, the invention provides the above method, wherein the longest step of the stepwise manner takes at least 3 minutes; and also the above method, wherein the longest step of the stepwise manner takes at least 10 minutes; and also the above method, wherein there is a transition time in between two consecutive pressures, and wherein the transition time is selected from a time that is at least 1, 2, 10, 20, 60, and 120 minutes; and also the above method, wherein each step of the stepwise manner takes at least 3 minutes or at least 10 minutes; and also the above method wherein the stepwise manner contains at least two steps; and also the above method, wherein the stepwise manner contains at least three steps.

In yet another embodiment that embraces microbes or proteins, what is provided is the above method, wherein the biologically active sample is a bacteria, a virus, a protein, an adjuvanted protein, or is a pharmaceutical antibody.

In a temperature embodiment, what is provided is the above method, wherein the mixture temperature, or shelf temperature is maintained at or above about 10 degrees C.; and also wherein the mixture temperature, or shelf temperature, is maintained at 15-25 degrees C.

In embodiments that delimit the nature of the microbe or microbes, what is provided is the above method, wherein the biologically active sample is bacteria, and wherein the bacteria used for the method are harvested in the stationary phase, and then used to form the mixture of the formulation and bacteria, and wherein the process stability of the cavitation-dried composition of bacteria is increased, where the increase in process stability is relative to that of a cavitation-dried composition, where bacteria are harvested in the log phase; and also the above method wherein the biologically active sample is bacteria, and wherein the bacteria used for the method are prepared by growing in hyperosmotic growth medium, and then used to form the mixture of the formulation and bacteria, and wherein the process stability of the cavitation-dried composition of bacteria is increased, where the increase in process stability is relative to that of a cavitation-dried composition where bacteria are prepared by growing in an iso-osmotic growth medium; and also the above method wherein the hyperosmotic growth medium contains 0.2-1.0 M NaCl.

In embodiments relating to steps, what is provided is the above method, wherein the pressure is decreased in a stepwise manner from about 10 Torr to less than about 100 mTorr; and also the above method, wherein there is a primary drying pressure, wherein the primary drying pressure used in cavitation drying process is reached within about three hours.

Polyol embodiments, polymer embodiments, plasticizer embodiments, amino acid embodiments, and the like include the following. What is provided is the above method of, wherein the polyol is 20% to 70% w/v of the mixture, and wherein the mixture contains a plasticizer that is 0.1% to 10.0% w/v of the mixture; and the above method, wherein the stability of the cavitation-dried composition is increased, relative to the stability of a cavitation-dried composition where the mixture is made by combining the biological sample with a formulation that does not contain a polyol; the above method, wherein the cavitation-dried composition contains a polymer, and is a polymer-containing composition, wherein the stability of the polymer-containing composition is increased, relative to a composition that contains all of the components of the polymer-containing composition but does not contain the polymer; and, in addition, the above method, wherein the polymer is one or more of gelatin, partially hydrolyzed gelatin, collagen, chondroitin sulfate, sialated polysaccharide, polyvinyl pyrrolidone, actin, myosin, microtubule protein, or serum albumin; and also the above method, wherein the formulation comprises 20-50% trehalose and 0-10% gelatin at pH 7-8; and also the above method, wherein the polyol is one or more of sucrose or trehalose.

In a methionine embodiment, what is embraced is the above method, wherein the formulation contains methionine, and wherein the methionine content of the mixture is about 0.5%, wherein the storage stability of the cavitation-dried composition is increased, relative to storage stability of a cavitation-dried composition where methionine is not in the formulation.

What is also embraced in the present invention is the above method, wherein the formulation contains a plasticizer, and wherein storage stability of the cavitation-dried composition is increased, relative to storage stability of a cavitation-dried composition wherein the plasticizer is not in the formulation; and also the above method, wherein the concentration of the plasticizer in the formulation is dimethylsulfoxide (DMSO), where the DMSO content of the mixture is about 0.5%-2.0% w/v, or glycerol, where the glycerol content of the mixture is about 0.5%-2.0% w/v; and also the above method, wherein the formulation contains gelatin, and wherein the gelatin content of the mixture is about 0.1-10% gelatin, and wherein the storage stability of the cavitation-dried composition is increased, relative to storage stability of a cavitation-dried composition wherein gelatin is not in the formulation.

In yet another microbial embodiment, what is embraces is the above method, wherein the biologically active sample is Salmonella, Shigella, Listeria, Franciscella, Escherichia coli, Pneumococcus, Mycobacterium, Pseudomonas, Staphylococcus, Streptococcus, or Bacillus anthracis.

In an embodiment that requires an additional step, what is provided is any of the above methods, where the method additionally comprises the step of mixing the formulation with the biologically active sample to form the mixture; and any of the above methods, wherein the method additionally comprises the step of harvesting the bacteria in the stationary phase; and any of the above methods, wherein the method additionally comprises the step of growing the bacteria in hyperosmotic growth medium.

In specific surface area embodiments, what is provided is any of the above compositions or methods, wherein the cavitation-dried composition has a specific surface area, and where the specific surface area is less than 0.3 meters squared per gram of mass; and also wherein the cavitation-dried composition has a specific surface area, and where the specific surface area is less than 0.1 meters squared per gram of mass.

What is also embraced are methods that provide films, and compositions that are films, wherein the cavitation-dried composition is a film.

In embodiments involving relative location of a surfactant, what is provided is compositions and methods, wherein the cavitation-dried composition contains a surfactant, and is a surfactant-containing composition, and wherein there is a ratio of residence of the biologically active sample at the surface of the surfactant-containing composition versus at the interior of the surfactant-containing composition, and wherein the surfactant results in a decrease in this ratio, as compared to the ratio in a second cavitation-dried composition that contains all of the components of the surfactant-containing composition but lacking the surfactant.

The present invention provides a cavitation-dried composition of a biological sample prepared according to any one of the embodiments disclosed above.

Formulations are also provided. The present invention embraces a formulation for preparing a cavitation-dried composition that comprises a biologically active sample, wherein the formulation contains a polyol (20-70% w/v), and a plasticizer (0.1-10.0% w/w), at pH 6.0-8.5; and also the above formulation, that is selected from a formulation of Tables 1, 2, 5, and 8.

Compositions and method of increased stability, that can be measured as stability to processing, stability to storage, or stability to the combination of processing and storage. For any optional component, or for any concentration that may be varied, stability of any “substance” is provided, as follows.

What is provided is any of the above compositions or methods, wherein the formulation contains a substance, and wherein the cavitation-dried composition is a substance-containing composition, and wherein the storage stability of the substance-containing composition is increased, relative to the storage stability of a cavitation-dried composition produced having the same components of the mixture used to make the substance-containing composition, except that the formulation does not contain substance.

### DEFINITIONS

Unless otherwise defined herein or below in the remainder of the specification, all technical and scientific terms used herein have meanings commonly understood by those of ordinary skill in the art to which the present invention belongs. Although any methods and materials similar or equivalent to those described herein can be used in the practice for testing of the present invention, the preferred materials and methods are described herein. In describing and claiming the present invention, the following terminology will be used in accordance with the definitions set out below.

Before describing the present invention in detail, it is to be understood that this invention is not limited to particular devices or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a component” can include a combination of two or more components; reference to “a buffer” can include mixtures of buffers, and the like.

Although many methods and materials similar, modified, or equivalent to those described herein can be used in the practice of the present invention without undue experimentation, the preferred materials and methods are described herein. In describing and claiming the present invention, the following terminology will be used in accordance with the definitions set out below.

The term “about”, as used herein, indicates the value of a given quantity can include quantities ranging within 10% of the stated value, or optionally within 5% of the value, or in some embodiments within 1% of the value.

“Ambient” temperatures or conditions are those at any given time in a given environment. Typically, ambient room temperature is 22° C., ambient atmospheric pressure, and ambient humidity are readily measured and will vary depending on the time of year, weather conditions, altitude, etc.

“Dry” in the context of dried compositions, as well as those prepared by freeze drying and spray drying, refers to residual moisture content less than about 10%. Dried compositions are commonly dried to residual moistures of 5% or less, or between about 3% and 0.1%.

“Excipients” or “protectants” (including cryoprotectants and lyoprotectants) generally refer to compounds or materials that are added to ensure or increase the stability of the therapeutic agent during the dehydration processes, e.g. foam drying, spray drying, freeze drying, etc., and afterwards, for long term stability.

“Glass” or glassy state” or “glassy matrix” refers to a liquid that has lost its ability to flow, i.e. it is a liquid with a very high viscosity, wherein the viscosity ranges from 10¹⁰ to 10¹⁴ pascal seconds. It can be viewed as a metastable amorphous system in which the molecules have vibrational motion but have very slow rotational and translational components. As a metastable system, it is stable for long periods of time when stored well below the glass transition temperature. Because glasses are not in a state of thermodynamic equilibrium, glasses stored at temperatures at or near the glass transition temperature relax to equilibrium and lose their high viscosity. The resultant rubbery or syrupy, flowing liquid is often chemically and structurally destabilized. While a glass can be obtained by many different routes, it appears to be physically and structurally the same material by whatever route it was taken. The process used to obtain a glassy matrix for the purposes of the invention is generally a solvent sublimation and/or evaporation technique.

The “glass transition temperature” is represented by the symbol T_{g} and is the temperature at which a composition changes from a glassy or vitreous state to a syrup or rubbery state. Generally T_{g} is determined using differential scanning calorimetry (DSC) and is standardly taken as the temperature at which onset of the change of heat capacity (Cₚ) of the composition occurs upon scanning through the transition. The definition of T_{g} is always arbitrary and there is no present international convention. The T_{g} can be defined as the onset, midpoint or endpoint of the transition.

A “stable” formulation or composition is one in which the biologically active material therein essentially retains its physical stability and/or chemical stability and/or biological activity upon storage. Stability can be measured at a selected temperature for a selected time period. Trend analysis can be used to estimate an expected shelf life before a material has actually been in storage for that time period.

“Pharmaceutically acceptable” refers to those active agents, salts, and excipients which are, within the scope of sound medical judgment, suitable for use in contact with the tissues or humans and lower animals without undue toxicity, irritation, allergic response and the like, commensurate with a reasonable benefit/risk ratio, and effective for their intended use.

### BRIEF DESCRIPTION OF THE DRAWINGS

FIG. 1 shows the growth curve of Ty21a, as measured by OD_{600 nm}, grown in either BHI broth with and without NaCl (▪) at various concentrations; 0.2M (), 0.3M (▴), and 0.4M (▾) NaCl.

FIG. 2 shows the effect of Ty21a's growth phase on its stability against process-related stress from drying; Ty21a titer prior to (▪) and post drying (□).

FIG. 3 shows the stability of cavitation-dried Ty21a stored at 25° C. in 1M trehalose; the symbols indicate starting material (black bars), post drying (white bars), 6 weeks of storage post drying at 25° C. (gray bars), and 16 weeks of storage post drying at 25° C. (dashed bars). The bacteria were grown in BHI with and without NaCl. The concentrations of NaCl in the growth media ranged from 0.1M to 0.6M NaCl. For all of the growth media conditions examined, Ty21a was cultured either in log phase or early stationary phase, as indicated in the figure.

FIG. 4 shows the titer loss associated with the cavitation process (white) and that following storage at 37° C. for 4 weeks (gray) for dried Ty21a formulated with and without excipients.

FIG. 5 shows the stability of cavitation-dried Ty21a stored at 25° C. in formulation 9 (▾), formulation 10 (), formulation 11 (▪), formulation 12 (♦), and formulation 13 (▴).

FIG. 6 shows the stability of cavitation-dried Ty21a in formulation 13 stored at 4° C. (▪), 25° C. (), 37° C. (▴), and 45° C. (▾).

FIG. 7 shows the stability of cavitation-dried Ty21a in formulation 12 stored at 4° C. (▪), 25° C. (), 37° C. (▴), and 45° C. (▾).

FIG. 8 shows the effect of formulation pH on the stability of cavitation-dried Ty21a in either formulation 9 or 12 stored at 25° C.; the symbols indicate starting material (black bars), post drying (white bars), and dried Ty21a after 2 weeks of storage (gray bars).

FIG. 9 shows the stability of cavitation-dried Ty21a-vectored Shigella sonnei vaccine in formulation 12 stored at 25° C.

FIG. 10 shows the stability of cavitation-dried Ty21a-vectored B. anthracis PA vaccine in formulation 9 stored at 25° C.

FIG. 11 shows the stability of freeze dried Ty21a in various formulations stored at 25° C.; the symbols indicate starting material (black bars), post-freeze drying (white bars), and freeze dried Ty21a after 1 week of storage (gray bars).

FIG. 12 shows the stability of spray dried Ty21a stored at 25° C. containing either sucrose, leucine, or sucrose and leucine; the symbols indicate starting material (▪), and spray dried Ty21a immediately after processing (□).

FIG. 13 shows the stability of spray dried Ty21a stored at 25° C. containing 3% (w/v) trehalose, 7% (w/v) sucrose, 0.02% (wt) Pluronic F68, 0.25% (wt) glycerol, and potassium phosphate adjusted to pH7.

FIG. 14 shows the stability of cavitation-dried Francisella in formulation Fr6 stored at 4° C. (▴), 25° C. (), and 37° C. (▪).

FIG. 15 shows the stability of cavitation-dried Ty21a in 30% (w/v) trehalose, 5% (w/v) gelatin, and 25 mM KPO₄ (pH8) stored at 4° C. (▴), 25° C. (), and 37° C. (▪).

FIG. 16. Storage stability at 37° C. of foam dried Ty21a containing varying amounts of methionine, ranging from 0-2% (w/v).

FIG. 17. Storage stability at 37° C. of foam dried Ty21a containing varying amounts of either (A) DMSO or (B) glycerol, both ranging in concentration from 0.5-2 wt %.

FIG. 18. Storage stability at 37° C. of foam dried Ty21a containing varying amounts of DMSO, ranging from 0-2 wt %. The base formulation contained 25% (w/v) trehalose, 0.5% (w/v) methionine, and 25 mM potassium phosphate buffer at pH8. Optimal storage stability was obtained for Ty21a foam dried in the presence of 1 wt % DMSO and 0.5% (w/v) methionine.

### DETAILED DESCRIPTION OF PREFERRED EMBODIMENTS

The present invention is the result of extensive experimentation to identify new combinations of vaccine formulation constituents and methods of preparing stable dry bacterial vaccines.

In one embodiment of dry Salmonella vaccine formulations, the viability of live attenuated bacteria is enhanced by selecting the bacteria from specific growth phase and formulation in the presence of specific combinations of pharmaceutically acceptable excipients dehydrated employing an optimized process. For example, the viability of dried Salmonella can be extended during storage at room temperature containing any of: 1) a polyol; 2) a soluble polymer; 3) an amino acid; 4) a plasticizer; 5) surfactants; and 6) a buffer at about pH 7.

Growth phase selection is described, as follows. Salmonella was found to be more resistant to drying process-induced activity loss when recovered from the early stationary growth phase than other growth phases (e.g. lag, logarithmic, late stationary).

Polyol embodiments of the present invention, without limitation, include the following. Salmonella was found to be more stable in the presence of a polyol, such as a substantially water soluble sugar. Furthermore, the polyol may be included to aid in certain drying processes, e.g. foam drying, by increasing the solution viscosity, and in freeze drying, by acting as a bulking agent. In addition, polyols can be included to modify the osmolarity of the Salmonella-containing solution to modify the transport of stabilizers across the bacterial membrane. In one aspect, the sugar is present in amount ranging from about 10% to 70% (w/v). In preferred embodiments, the sugar is present in the formulation in the range between 10% and 70%, 20% and 60%, 30% and 50%, or about 40% (w/v). In preferred embodiments the sugars are present in the formulations at a concentration ranging from about 20% to about 40% (w/v).

More preferred polyols include, e.g., sucrose, trehalose, sorbose, melezitose, sorbitol, stachyose, raffinose, fructose, mannose, maltose, lactose, arabinose, xylose, ribose, rhamnose, galactose, glycose, mannitol, xylitol, erythritol, threitol, sorbitol, glycerol, L-glyconate, dextrose, fucose, polyaspartic acid, inositol hexaphosphate (phytic acid), sialic acid , N-acetylneuraminic acid-lactose, and their combinations thereof. In a typical embodiment, the formulation sugar is a monosaccharide or a disaccharide. In preferred embodiments, the sugar is trehalose ranging in concentration between about 20% to about 50% (w/v), or to 20% to 50% (w/v).

Polymer embodiments of the present invention, without limitation, include the following. Formulations of the present invention appear to benefit from the presence of a polymer in the formulation. Similar to polyols, polymers can be included to increase the solution viscosity and to provide structural strength during a drying process, e.g. foam drying and freeze drying. In case of spray drying, polymers can be included to modify the surface properties of atomized droplets. In preferred embodiments, the polymer is ingestible. Preferably, the polymer has significant ionic character, preferably anionic character. In certain embodiments, the polymer is present in a concentration ranging from about 0.1% to 20% (w/v), or about 5% (w/v).

More preferred polyols include, e.g., gelatin, hydrolyzed gelatin, collagen, chondroitin sulfate, a sialated polysaccharide, water soluble polymers, polyvinyl pyrrolidone, actin, myosin, microtubules, dynein, kinetin, bovine serum albumin, human serum albumin, and their combinations thereof. In one embodiment, the polymer is gelatin. In certain embodiments, the formulation comprises 5% (w/v) gelatin or hydrolyzed gelatin.

Amino acid embodiments of the present invention, without limitation, include the following. Amino acids can help stabilize bacterial membrane structures and contribute to pH buffering. Amino acids can also be useful in modifying the osmolarity of the Salmonella-containing solution, the solution pH, and the surface tension of solution during processing, e.g. foam drying and spray drying. In some embodiments of the invention, amino acids are present in the formulation in amounts ranging from about 0.1% to 5% (w/v). In preferred embodiments, one or more amino acids are present at a concentration ranging from 0.5% to 1.5% (w/v), or about 1% (w/v).

Preferred amino acids for incorporation into the inventive formulations are, e.g., alanine, arginine, methionine, serine, lysine, histidine, glutamic acid, and/or the like. In a most preferred embodiment, the amino acid is methionine, e.g., at a concentration near 1% (w/v).

Plasticizer embodiments of the present invention, without limitation, encompass the following. Formulations of the present invention appear to benefit from the presence of a plasticizer in the formulation. In some embodiments of the invention, plasticizers are present in the formulation in amounts ranging from about 0.1% to 5% by weight of said formulation. In preferred embodiments, one or more plasticizers are present at a concentration ranging from 0.5% to 3%, or about 2% by weight of said formulation.

More preferred plasticizers include, e.g., glycerol, dimethylsulfoxide (DMSO), propylene glycol, ethylene glycol, oligomeric polyethylene glycol, sorbitol, and their combinations thereof. In one embodiment, the plasticizer is glycerol. In certain embodiments, the formulation comprises about 2% glycerol by weight of said formulation.

Surfactant embodiments of the invention, without limitation, encompass the following. Formulations of the present invention appear to benefit from the presence of a surfactant in the formulation. Furthermore, the surfactant may be included to aid in certain drying processes, e.g. foam drying and spray drying, by decreasing the surface tension and in coating the particle surface, respectively. Surfactants may also be included to enhance the solubility of other formulation constituents. In certain embodiments, the polymer is present in a concentration ranging from about 0.001% to 2% by weight of said formulation, or about 0.2%.

More preferred surfactants include, e.g., Tween20®, Span20®, Tween80®, Pluronic® poloxamer, polyethylene glycol, polypropylene glycol, polyethylene glycol/polypropylene glycol block copolymers, polyethylene glycol alkyl ethers, polyethylene glycol sorbitan monolaurate, polypropylene glycol alkyl ethers, polyethylene glycol/polypropylene glycol ether block copolymers, polyoxyethylenesorbitan monooleate, alkylarylsulfonates, phenylsulfonates, alkyl sulfates, alkyl sulfonates, alkyl ether sulfates, alkyl aryl ether sulfates, alkyl polyglycol ether phosphates, polyaryl phenyl ether phosphates, alkylsulfosuccinates, olefin sulfonates, paraffin sulfonates, petroleum sulfonates, taurides, sarcosides, fatty acids, alkylnaphthalenesulfonic acids, naphthalenesulfonic acids, lignosulfonic acids, condensates of sulfonated naphthalenes with formaldehyde and phenol, lignin-sulfite waste liquor, alkyl phosphates, quaternary ammonium compounds, amine, oxides, betaines, and/or the like. In one embodiment, the surfactant is block copolymers of polyethylene and polypropylene glycol. In certain embodiments, the formulation comprises about 0.2% block copolymers of polyethylene and polypropylene glycol by weight of said formulation.

Buffer embodiments of the present invention, without implying any limitation, encompass the following. Pharmaceutically acceptable buffering components are included in the present invention to adjust the pH and the osmolarity of the formulation. In some embodiments of the invention, buffers are present in the formulation in concentration ranging from about 5 mM to 2M. In preferred embodiments, one or more buffering components are present at a concentration ranging from about 5 mM to about 100 mM, or at about 25 mM.

Preferred buffers for incorporation into the inventive formulations are, e.g., potassium phosphate, sodium phosphate, sodium acetate, histidine, imidazole, sodium citrate, sodium succinate, ammonium bicarbonate, a carbonate, and/or the like. In a most preferred embodiment, the buffer is potassium phosphate, e.g., at a concentration near 25 mM.

Typically, the pH of the inventive formulation is adjusted to provide a physiological pH, such as pH7.4, a pH ranging from about pH 4 to about pH9, from pH 5 to pH 8, or about pH 7.

Buffering capacity of the current invention can be provided by the buffer or an amino acid, if included.

Preferred combinations of excipient constituents to stabilize dried formulations of a live attenuated Salmonella vaccine include, e.g., combinations of a sugar and potassium phosphate buffer at about pH 7. In more preferred embodiments, the sugar can be trehalose at a concentration ranging from about 20% to about 40% (w/v), or about 25% (w/v). In yet another embodiment of the present invention, the sugar can be sucrose at a concentration ranging from about 20% to about 40% (w/v), or about 25% (w/v).

In addition to the above combinations of constituents, it can be beneficial to include an amino acid, such as methionine, e.g., at a concentration ranging from about 0.5% to about 2% (w/v), or about 1% (w/v).

In addition to the combinations of constituents described above, it can be beneficial to include a plasticizer, such as glycerol and DMSO, e.g., at a concentration ranging from about 0.5% to about 3% by weight of said formulation, or about 2%. In more preferred embodiments, the plasticizer can be glycerol at a concentration ranging from about 0.5% to about 3% by weight of said formulation, or about 2%.

Furthermore, it can be beneficial to include a polymer to the combination of constituents described above, such as gelatin, e.g., at a concentration ranging from about 1% to about 10% (w/v). In more preferred embodiments, the polymer can be gelatin at a concentration of about 5% (w/v).

In the present inventive formulations, the presence of surfactants can possibly enhance stability. Furthermore, the choice of processing method to dry Salmonella may dictate the use of surfactants, e.g., foam drying and spray drying. In more preferred embodiments, the surfactant can be block copolymers of polyethylene and polypropylene glycol at a concentration ranging from about 0.02% to 1% by weight of said formulation, or about 0.2%.

Dry powder production embodiments of the invention are provided. In another aspect of the present invention, the Salmonella-containing compositions may be prepared as a dry powder. Dry powder production can be conducted employing a variety of methods known to those skilled in the art, which includes, but is not limited to foam drying, freeze drying, spray drying, spray freeze drying, fluidized bed drying, supercritical fluid assisted drying, and vacuum drying. For the present invention, a cavitation method conducted using a freeze-dryer was employed.

In one embodiment, a cavitation method comprises of gradually decreasing the freeze-dryer chamber pressure from atmospheric pressure or ambient pressure to approximately 100 mTorr in 5 to 90 minutes, while maintaining the shelf-temperature at a value in between −10° C. to 40° C. “Shelf temperature” refers to the temperature inside the freeze-dryer, at the shelf that supports the samples. In a preferable method, the freeze dryer chamber pressure is reduced stepwise from ambient pressure to 50 Torr, to 4 Torr, to 1 Torr, and to 100 mTorr within 90 minutes. In another embodiment of the method, the process may further comprise of holding the reduced pressure and drying temperature for a time ranging from about 12 hours to about 5 days. In a preferred embodiment, the reduced pressure and drying temperature are held for about 48 hours. The foaming process is dependent on the composition of the formulation, particularly the viscosity and the surface tension of the solution. If the solution concentration is not high enough (thus the requirement on high solids content) the solution will not foam or “cavitate”. For difficult to foam samples, the inclusion of surfactants can help the foaming or “cavitation” process.

Regarding ramping, a ramping procedure and ramping rate involve decreasing (or increasing) the pressure from a first pressure to a second pressure, where equilibrium is not reached, where this equilibrium refers to movement of solvent molecules from the sample to the gaseous phase, and from the gaseous phase to the sample. In ramping, during the transition from a first higher pressure to a second lower pressure, substantial evaporation from the sample does not occur. What is provided is a ramping procedure that is more than 10 minutes, greater than 15 minutes, longer than 20 minutes, greater than 30 minutes, or more than 40 minutes, and the like. In a transition from a first pressure that is higher, to a second pressure that is substantially lower, a quick transition can cause freezing, while ramping can avoid or prevent freezing. An instantaneous transition from a first pressure that is higher to a second pressure that is substantially lower can cause freezing of the sample.

The invention provides a method for preparing a cavitation-dried formulation of bacteria, from a mixture of a formulation and bacteria, comprising decreasing the chamber pressure in a stepwise manner to reduce water content of the mixture, wherein a foam is produced, and wherein the mixture does not freeze and the foam does not freeze, and where prior to decreasing the chamber pressure, the bacteria had been harvested at stationary phase.

The invention avoids freezing. By avoiding freezing, the invention reduces shearing, and reduces bursting of bubbles during foam formation.

What is provided is a formulation, where the mass fraction of plasticizer/polyol is 5%. In another aspect, what is provided is a formulation where the mass fraction of plasticizer/polyol is about 1%, or approximately 2%, or approximately about 3%, or about 4%, or around 5%, or approximately 6%, or approximately about 8%, or around 10%, or the like. In a formulation embodiment, the invention encompasses a formulation, either as an independent composition of matter, or as combined with a biologically active compound, and the like, that is selected from a formulation of Table 1, 2, 3, 4, 5, 6, 7, and 8, and the like, or selected from a formulation of any combination of these tables, or selected from only one individual table.

The invention provides a method for preparing a cavitation-dried formulation of bacteria, from a mixture of a formulation and bacteria, comprising decreasing the air pressure in a stepwise manner to reduce water content of the mixture, wherein a foam is produced, and wherein the mixture does not freeze and the foam does not freeze, and where prior to decreasing the chamber pressure, the method further comprises the step of transferring the bacteria to the formulation to form the mixture.

The invention provides a method for preparing a cavitation-dried formulation of bacteria, from a mixture of a formulation and bacteria, comprising decreasing the chamber pressure in a stepwise manner to reduce water content of the mixture, wherein a foam is produced, and wherein the mixture does not freeze and the foam does not freeze, and where prior to decreasing the air pressure, the method further comprises the steps of harvesting the bacteria from the stationary phase, and transferring the bacteria to the formulation to form the mixture.

The invention provides a method for preparing a cavitation-dried formulation of bacteria, from a mixture of a formulation and bacteria, comprising decreasing the chamber pressure in a stepwise manner to reduce water content of the mixture, wherein a foam is produced, and wherein the mixture does not freeze and the foam does not freeze, and where prior to decreasing the air pressure, the method further comprises the steps of growing the bacteria in a hyperosmotic medium, such as a medium containing 0.2-0.4M NaCl, harvesting the bacteria from the stationary phase, and transferring the bacteria to the formulation to form the mixture.

In another aspect, the invention provides a method, and composition made by this method, wherein the cavitation-dried composition contains a polymer, and wherein the stability of the cavitation-dried composition is increased, in that the loss of titer during processing or storage is reduced by at least 10%, reduced by at least 20%, or reduced by at least 30%, relative to a cavitation-dried composition where the formulation does not contain the polymer, and where the stability is process stability or storage stability, or the combination of process and storage stability.

Moreover, what is contemplated, is a method, and a composition made by this method, wherein the cavitation-dried composition contains a polyol, and wherein the stability of the cavitation-dried composition is increased, in that the loss of titer during processing or storage is reduced by at least 10%, reduced by at least 20%, or reduced by at least 40%, relative to a cavitation-dried composition where the formulation does not contain the polyol, and where the stability is process stability or storage stability, or the combination of process and storage stability.

In still another aspect, the invention comprises, is a method, and a composition made by this method, wherein the cavitation-dried composition contains an amino acid, and wherein the stability of the cavitation-dried composition is increased, in that the loss of titer during processing or storage is reduced by at least 10%, reduced by at least 20%, or reduced by at least 40%, relative to a cavitation-dried composition where the formulation does not contain the amino acid, and where the stability is process stability or storage stability, or the combination of process and storage stability.

In yet another embodiment, what is contemplated, is a method, and a composition made by this method, wherein the cavitation-dried composition contains a plasticizer, and wherein the stability of the cavitation-dried composition is increased, in that the loss of titer during processing or storage is reduced by at least 10%, reduced by at least 20%, or reduced by at least 40%, relative to a cavitation-dried composition where the formulation does not contain the plasticizer, and where the stability is process stability or storage stability, or the combination of process and storage stability.

In these aspects and embodiments, where the bioactive material or immunologically active material is a bacteria, virus, enveloped virus, non-enveloped virus, microbe, or microorganism, the stability can be measured by titer. Moreover, for any bioactive material, the stability that is measured can be process stability (the process of making the dried foam), or it can be storage stability, or it can be the combined stability of process stability and storage stability.

Adjuvants embodiments are also provided by the present invention, including aluminum adjuvants and calcium adjuvants. See, e.g., U.S. Pat. No. 5,773,007 issued to Penney, and U.S. Pat. No. 6,610,308 issued to Haensler. What is also encompassed are adjuvants that are toll-like receptor agonists (TLR agonists), including oligonucleotides. In the present invention, the TLR agonist can be covalently bound to the biologically active sample, or it can be non-covalently bound to the biologically active sample, or it can be merely mixed with (and not attached to) the biologically active sample.

The biologically active sample of the present invention can be a human protein, such as a human antibody, cytokine, or hormone, that does not elicit immune responses against itself when administered to a human subject. Also, the biologically active sample can be a protein, of human or non-human origin, that has been engineered or chemically altered so that it does not elicit immune responses against itself when administered to a human subject. Pharmaceutically useful examples include humanized recombinant biologicals, such as antibodies.

Foaming usually involves the rising of bubbles, but does not necessarily encompass bursting bubbles. In contrast, cavitation encompasses the rising and bursting of bubbles. Cavitation can produce a foam. Cavitation can produce a film.

Cavitation-dried compositions have less specific surface area than a typical freeze-dried cake. A composition with a lesser specific surface area has the advantage, where the composition is a protein, cell, or an oxygen-sensitive substance, of reducing exposure to air. Air can facilitate the denaturation of proteins, because air is not a hydrophilic substance, and air that contains oxygen can enhance oxidative damage.

The polyol in the compositions of the present invention can influence the specific surface area. If the polyol concentration is too low, bubble-bursting may be too violent and uncontrolled, resulting in: (1) Loss of product from the container, (2) Causing product to touch the sealant or rubber stopper; (3) Causing incorrect structure of foam in the final structure, for example, violent bubbling can results in a very low specific surface area. Specific surface area can be calculated from the surface area divided by the weight of the material.

Films are also encompassed by the present invention. A film has a relatively low specific surface area. Films have a relatively low specific surface area, that is, lower than those of freeze-dried cakes and of foams. U.S. Pat. No. 7,229,645 issued to Maa, et al., and U.S. Pat. No. 6,284,282 issued to Maa and Nguyen, provides information on specific surface area.

The present invention also contemplates compositions, and related methods, that are not crushed, that are not mechanically crushed, or that are not substantially crushed.

The present invention provides formulations, mixtures, and methods, that encompass cavitation-dried compositions, where the specific surface area is within a preferred range.

The preferred range of the specific surface area is often less than 1.0 meters squared per gram of mass, typically less than 0.8 meters squared per gram of mass, often less than 0.6 meters squared per gram of mass, usually less than 0.4 meters squared per gram of mass, often less than 0.3 meters squared per gram of mass, sometimes less than 0.2 meters squared per gram of mass, frequently less than 0.1 meters squared per gram of mass, and the like.

Specific surface area can be measured by the Brunauer, Emmet, and Teller (BET) method of SSA measurement, using either nitrogen or krypton gas, and measuring the absorption of the gas onto the surface of the solids, as one permeates the gas through the pores of the pharmaceutical solids. See, e.g., Costantino H R, Curley J G, Hsu C C. (1997) Determining the water sorption monolayer of lyophilized pharmaceutical proteins. J. Pharm. Sci. 86:1390-1393; Hsu C C, et al. (1992) Determining the optimum residual moisture in lyophilized protein pharmaceuticals. Dev. Biol. Stand. 74:255-270.

Without implying any limitation, the compositions and methods of the present invention, include the following advantages. With regard to films, what is provided are compositions and methods with a practical lower limit of specific surface area that is sometimes 0.01, often 0.008, occasionally 0.006, typically 0.004, sometimes 0.002, and most preferably 0.001 meter squared per gram, for a cavitation-dried film. With regard to foams, what is provided are compositions and methods with a practical lower limit of specific surface area that is sometimes 0.1, often 0.08, occasionally 0.06, or 0.05, typically 0.04, or 0.03, sometimes 0.02, and most preferably 0.01 meter squared per gram, for a cavitation-dried foam.

Another advantage of the present invention, is that the cavitation-dried compositions of the present invention, compared to compositions prepared by other methods, result in lower ratios of [surface]/[interior], as it applies to the location of the biologically active substance, e.g., protein. When a surfactant is included in the mixture of formulation and biologically active substance, for example, a protein, the resulting cavitation-dried composition is configured so that the surfactant occupies the surface and tends to exclude the protein from the surface, resulting in a greater proportion of the protein in the interior. This advantage applies to both cavitation-dried foams and cavitation-dried films. Surface coverage can be determined by electron spectroscopy chemical analysis (ESCA).

Yet another advantage of the compositions and methods of the present invention, is use of limited amounts, that is, amounts in a relatively narrow range or in a controlled range, of plasticizer. The addition of greater amounts of plasticizer, or unlimited amounts of plasticizer, can result in a product of less than maximal stability.

In the steps used in cavitation-drying, without intending any limitation, the time for a pressure drop can be 10-20 seconds, 20-30 seconds, 30-40 seconds, 40-60 seconds, as well as 30-60 seconds, 60-90 seconds, 90-120 seconds, 120-150 seconds, 150-180 seconds, 180-210 seconds, 210-240 seconds, 240-270 seconds, 270-300 seconds, 300-330 seconds, 330-360 seconds 390 seconds, 390-420 seconds, 420-450 seconds, 450-480 seconds, 480-510 seconds, 510-540 seconds, 540-570 seconds, 570-600 seconds, and the like.

In the steps used in cavitation-drying, without limitation, the time for holding at a particular pressure can be 10-20 seconds, 20-30 seconds, 30-40 seconds, 40-60 seconds, as well as 30-60 seconds, 60-90 seconds, 90-120 seconds, 120-150 seconds, 150-180 seconds, 180-210 seconds, 210-240 seconds, 240-270 seconds, 270-300 seconds, 300-330 seconds, 330-360 seconds 390 seconds, 390-420 seconds, 420-450 seconds, 450-480 seconds, 480-510 seconds, 510-540 seconds, 540-570 seconds, 570-600 seconds, and the like.

In the steps used in cavitation-drying, without limitation, the pressure at a step can be, for example, about 600 Torr, about 500 Torr, about 400 Torr, about 300 Torr, about 200 Torr, about 100 Torr, about 80 Torr, about 60 Torr, about 40 Torr, about 20 Torr, about 15 Torr, about 10 Torr, about 5 Torr, about 4 Torr, about 3 Torr, about 2 Torr, about 1 Torr, about 0.8 Torr, about 0.6 Torr, about 0.4 Torr, about 0.2 Torr, about 0.1 Torr, about 0.08 Torr, about 0.06 Torr, about 0.04 Torr, and the like.

### Examples

The following examples are offered to illustrate, but not to limit the scope of the claimed invention.

### Example 1

### Effect of Ty21a Growth Media on Growth Kinetics

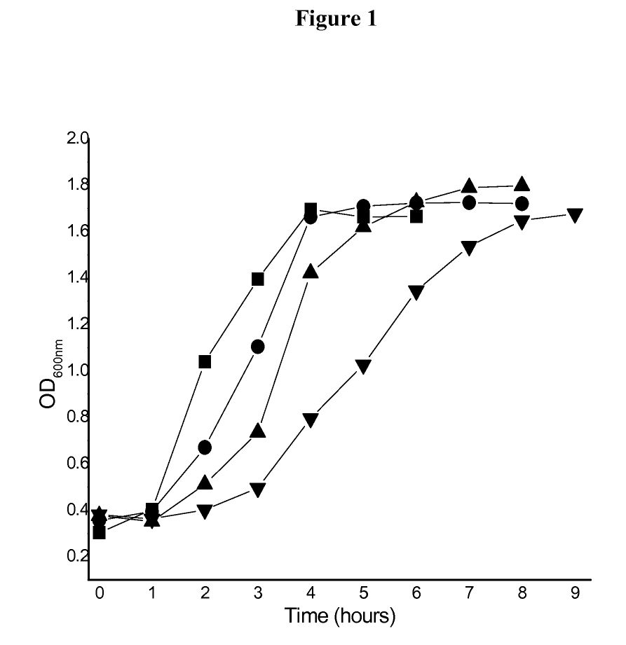

Ty21a was cultured by inoculation in BHI broth and also in BHI broth containing 0.2, 0.3, or 0.4M NaCl. The bacterial suspension was shaken at 220 rpm while the temperature was maintained at 37° C. The kinetics of bacterial growth was monitored at OD_{600 nm} (FIG. 1). The volume of the culture was about 50 mL, and the flask volume was about 500 mL. The term “early stationary phase” refers to a culture at the time immediately after the optical density (Abs₆₀₀) has reached a plateau value, while the term “stationary phase” refers to a culture at later times.

### Example 2

### Effect of Ty21a Growth Phase on Process Recovery

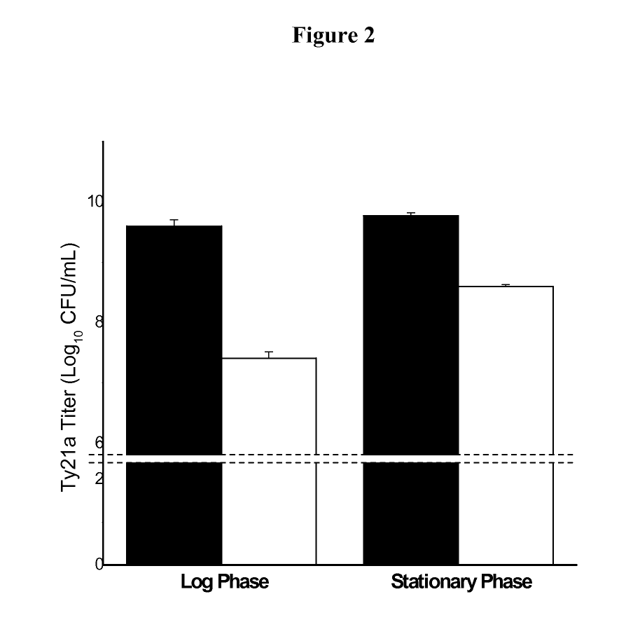

Live attenuated Salmonella enterica Serovar Typhi vaccine strain, Ty21a, was cultured by inoculation in brain heart infusion (BHI) broth overnight and was harvested in both log phase (1.6 OD_{600 nm}) and early stationary phase (2.2 OD_{600 nm}). The samples were centrifuged at 2500 rcf for 10 minutes, and the resulting bacterial pellet was resuspended in 1M trehalose and taken to the initial volume. 0.5 mL aliquot of the bacterial sample was placed into individual vials and dried according to cycle 1: 1) 15° C. at atmospheric pressure for 10 min, 2) 15° C. at or below 50 mTorr for 24 hours, and 3) 33° C. at or below 50 mTorr for 24 hours. The samples were reconstituted with double-filtered deionized water and plated out on (trypticase soy broth) TSB plates to determine viability (FIG. 2). The plates were counted after 16 hours of incubation at 37° C. In the above example, the pressure was decreased gradually, over the course of several minutes, in the transition from atmospheric pressure down to 50 mTorr.

### Example 3

### Effect of Ty21a Growth Media on Process Recovery

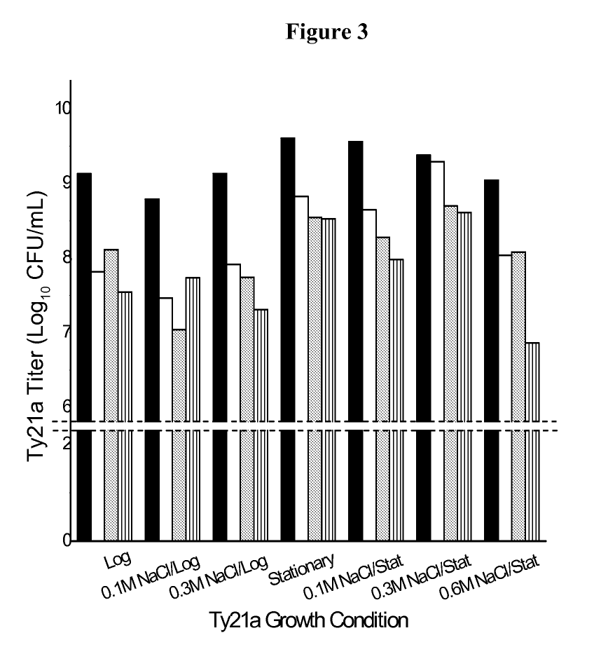

Ty21a was cultured by inoculation in BHI broth and also in BHI broth containing 0.1, 0.3, or 0.6M NaCl. Bacteria grown in each broth were harvested in both log phase (1.6 OD_{600 nm}) and early stationary phase (2.2 OD_{600 nm}). The samples were centrifuged at 2500 rcf for 10 minutes, and the resulting bacterial pellet was resuspended in 1M trehalose and taken to the initial volume. 0.5 mL aliquot of the bacterial sample was placed into individual vials and dried according to cycle 1. The vials were sealed under slight vacuum (˜650 Torr) in argon gas, crimped, and stored at 25° C. The vials were taken out at various time points and then reconstituted with double-filtered deionized water. The samples were plated out on TSB plates for viability determination (FIG. 3). The plates were counted after 16 hours of incubation at 37° C.

### Example 4

### Effect of Formulation Components on the Process Recovery of Cavitation-Dried Ty21a

Ty21a was cultured by inoculation in BHI broth overnight and was harvested in the early stationary phase (2.2 OD_{600 nm}). The sample was centrifuged at 2500 rcf for 10 minutes, and the resulting bacterial pellet was resuspended in the formulations shown below in Table 1 and taken to the initial volume. 0.5 mL aliquot of the bacterial sample was placed into individual vials and dried according to cycle 2: 1) 15° C. at atmospheric pressure for 10 minutes, 2) 15° C. at 10 Torr for 10 minutes, 3) 15° C. at 4 Torr for 10 minutes, 4) 15° C. at 1 Torr for 10 minutes, 5) 15° C. at 100 mTorr for 42 hr, and 6) 20° C. at 100 mTorr for 22 hr. The vials were sealed under slight vacuum (˜650 Torr) in argon gas, crimped, and stored at 25° C. The dried samples were reconstituted with double-filtered deionized water and were plated out on TSB plates to determine the process-associated loss in bacterial titer (Table 1). The plates were counted after 16 hours of incubation at 37° C.

TABLE 1
Formulation Compositions for Cavitation-Dried Ty21a

| Components | 1 | 2 | 3 | 4 | 5 | 6 | 7 | 8 |
|---|---|---|---|---|---|---|---|---|
| Trehalose | 30 | 30 | 25 | 25 | 25 | 25 | 25 | 25 |
| (%, w/v) | | | | | | | | |
| Sucrose | | | | | | | | |
| (%, w/v) | | | | | | | | |
| Gelatin | | | | | | | 5 | 5 |
| (%, w/v) | | | | | | | | |
| Pluronic F68 | | | | | | | | 0.2 |
| (wt %) | | | | | | | | |
| Methionine | | | 0.5 | 0.5 | 0.5 | 0.5 | 1 | 2 |
| (%, w/v) | | | | | | | | |
| Glycerol | | | | | | 2.4 | 2.4 | 2.4 |
| (wt %) | | | | | | | | |
| DMSO (wt %) | | | | 0.5 | 2.4 | | | |
| KPO₄ (mM) | 25 | 25 | 25 | 25 | 25 | 25 | 25 | 25 |
| pH | 7 | 8 | 8 | 8 | 8 | 7 | 7 | 7 |
| Process Loss | 0.25 | 0.40 | 0.45 | 0.35 | 0.12 | 0.32 | 0.89 | 0.97 |
| (Log₁₀) | | | | | | | | |

### Example 5

### Effect of Formulation Components on the Stability of Cavitation-Dried Ty21a

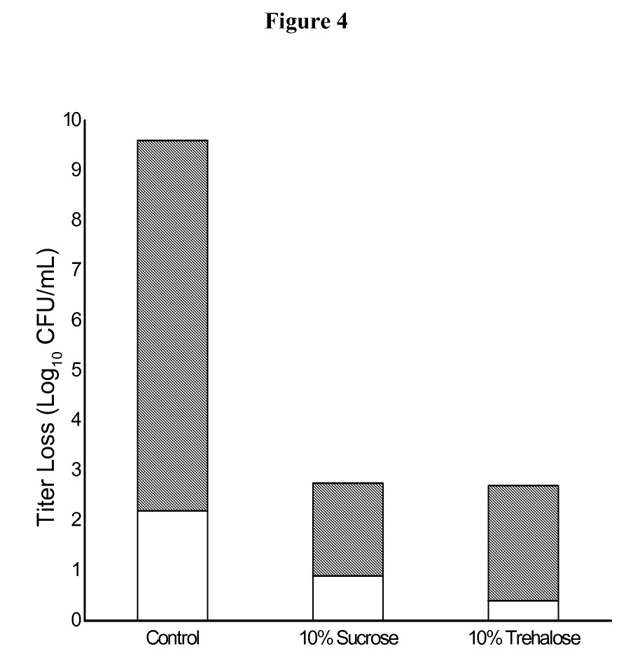

Ty21a was cultured by inoculation in BHI broth overnight and was harvested in the stationary phase (2.2 OD_{600 nm}). The sample was centrifuged at 2500 rcf for 10 minutes, and the resulting bacterial pellet was resuspended in 10% (w/v) sucrose, 10% (w/v) trehalose, or in water, and taken to the initial volume. 0.5 mL aliquot of the bacterial sample was placed into individual vials and dried according to cycle 1. The vials were sealed under slight vacuum (˜650 Torr) in argon gas, crimped, and stored at 37° C. The vials were taken out after 4 weeks of storage and then reconstituted with double-filtered deionized water. The samples were plated out on TSB plates for viability determination (FIG. 4). The plates were counted after 16 hours of incubation at 37° C. To provide further commentary regarding the method of this example, the drying was conducted following cycle 1 and then prior to removing the vials (after completion of cavitation drying), the pressure was increased to a value slightly-below ambient (650 Torr). This process of back-fill (using non humidified, inert gas) was conducted for all samples following processing. The purpose of this process is to reduce the influx of moisture or other gasses during storage, which would have otherwise entered the sealed vial (due to the negative pressure maintained within the vial).

### Example 6

### Effect of Formulation Components on the Stability of Cavitation-Dried Ty21a

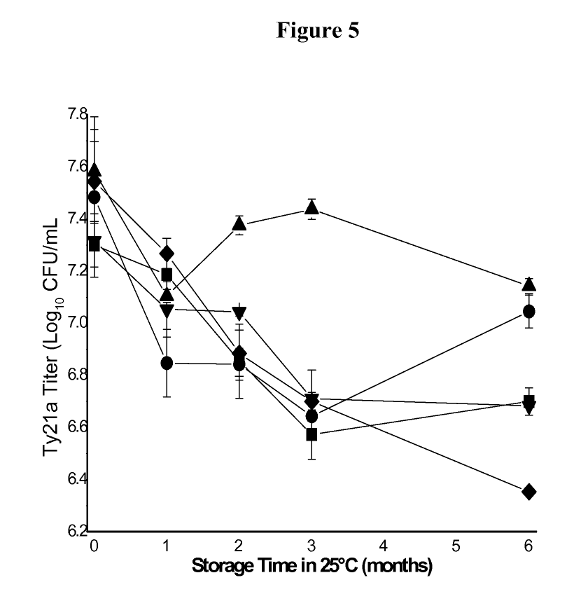

Ty21a was cultured by inoculation in BHI broth overnight and was harvested in the stationary phase (2.2 OD_{600 nm}). The sample was centrifuged at 2500 rcf for 10 minutes, and the resulting bacterial pellet was resuspended in the formulations shown below in Table 2 and taken to the initial volume. 0.5 mL aliquot of the bacterial sample was placed into individual vials and dried according to cycle 1. The vials were sealed under slight vacuum (˜650 Torr) in argon gas, crimped, and stored at 25° C. The vials were taken out at various time points and then reconstituted with double-filtered deionized water. The samples were plated out on TSB plates for viability determination (FIG. 5). The plates were counted after 16 hours of incubation at 37° C.

TABLE 2
Formulation Compositions for Cavitation-Dried Ty21a

| Components | 9 | 10 | 11 | 12 | 13 |
|---|---|---|---|---|---|
| Trehalose (%, w/v) | | | | 25 | 25 |
| Sucrose (%, w/v) | 40 | 40 | 40 | | |
| Gelatin (%, w/v) | | | 5 | | 5 |
| Pluronic F68 (wt %) | | | 0.2 | | 0.2 |
| Methionine (%, w/v) | | 1 | 1 | 1 | 1 |
| Glycerol (wt %) | 2.1 | 1 | 2.4 | 2.1 | 2.4 |
| KPO₄ (mM) | 25 | 25 | 25 | 25 | 25 |
| pH | 7 | 7 | 7 | 7 | 7 |

### Example 7

### Stability of Cavitation-Dried Ty21a at Various Temperatures

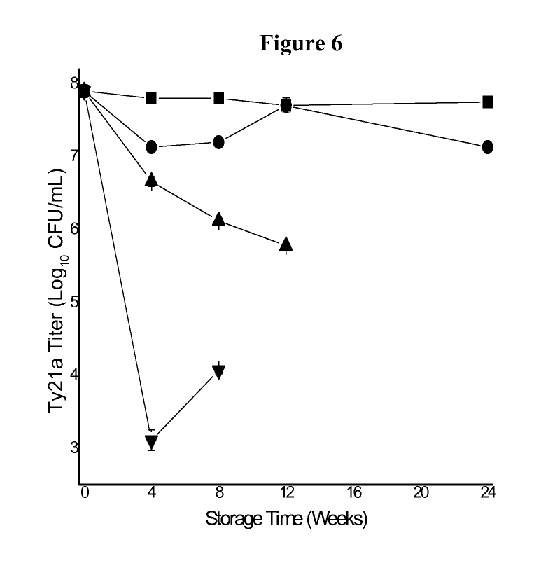

Live attenuated Salmonella enterica Serovar Typhi vaccine strain, Ty21a, was cultured by inoculation in BHI broth overnight and harvested in the stationary phase (2.2 OD_{600 nm}). The sample was centrifuged at 2500 rcf for 10 minutes, and the resulting bacterial pellet was resuspended in formulation 13 (Table 2) and taken to the initial volume. 0.5 mL aliquot of the bacterial sample was placed into individual vials and dried according to cycle 1. The vials were sealed under slight vacuum (˜650 Torr) in argon gas, crimped, and stored at various temperatures including 4, 25, 37, and 45° C. The vials were taken out at various time points and then reconstituted with double-filtered deionized water. The samples were plated out on TSB plates for viability determination (FIG. 6). The plates were counted after 16 hours of incubation at 37° C.

### Example 8

### Stability of Cavitation-Dried Ty21a at Various Temperatures

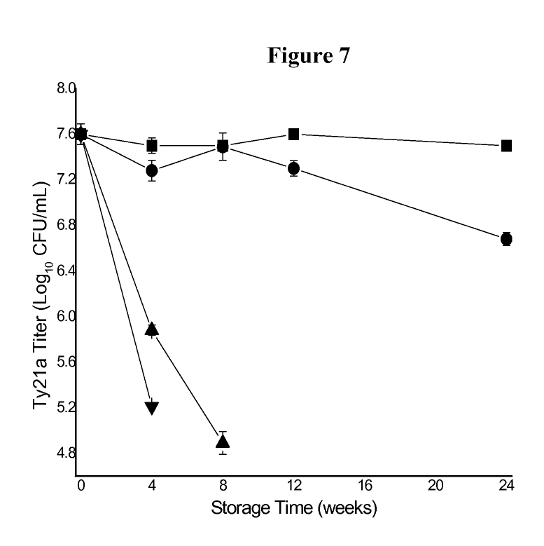

Ty21a was cultured by inoculation in BHI broth overnight and harvested in the stationary phase (2.2 OD_{600 nm}). The sample was centrifuged at 2500 rcf for 10 minutes, and the resulting bacterial pellet was resuspended in formulation 12 (Table 2) and taken to the initial volume. 0.5 mL aliquot of the bacterial sample was placed into each vial and dried according to cycle 1. The vials were sealed under slight vacuum (˜650 Torr) in argon gas, crimped, and stored at various temperatures including 4, 25, 37, and 45° C. The vials were taken out at various time points and then reconstituted with double-filtered deionized water. The samples were plated out on TSB plates for viability determination (FIG. 7). The plates were counted after 16 hours of incubation at 37° C.

### Example 9

### Effect of pH on the Stability of Cavitation-Dried Ty21a

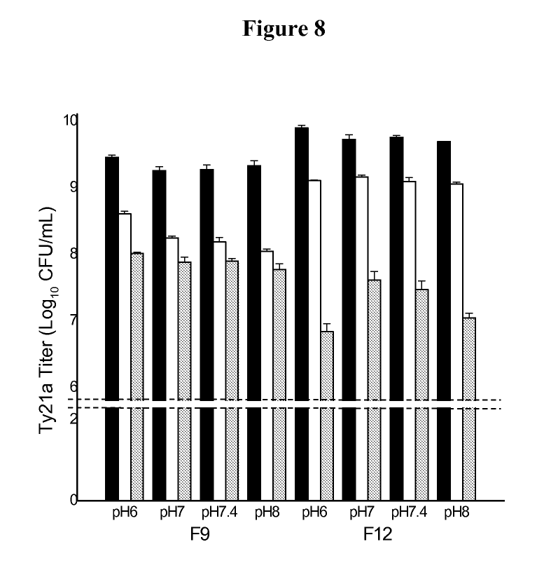

Ty21a was cultured by inoculation in BHI broth overnight and harvested in the stationary phase (2.2 OD_{600 nm}). The sample was centrifuged at 2500 rcf for 10 minutes, and the resulting bacterial pellet was resuspended in either formulation 9 or 12 (Table 2), prepared at pH values ranging from pH6 to pH8, and taken to the initial volume. 0.5 mL aliquot of the bacterial sample was placed into individual vials and dried according to cycle 3: 1) −5° C. at atmospheric pressure for 15 min, 2) −5° C. at 1 Torr for 30 minutes, 3) 0° C. at 1 Torr for 30 minutes, 4) 10° C. at 1 Torr for 30 minutes, 5) 10° C. at or below 50 mTorr for 48 hours, and 6) 4° C. at or below 50 mTorr for 24 hours. Samples were plated out on TSB plates immediately following drying. The plates were counted after 16 hours of incubation at 37° C. The vials following drying were sealed under slight vacuum (˜650 Torr) in argon gas, crimped, and stored at 25° C. The vials were taken out after two weeks and then reconstituted with double-filtered deionized water and plated out on TSB plates for viability determination (FIG. 8).

### Example 10

### Stability of Cavitation-Dried Ty21a-Vectored Shigella sonnei Vaccine at Room Temperature

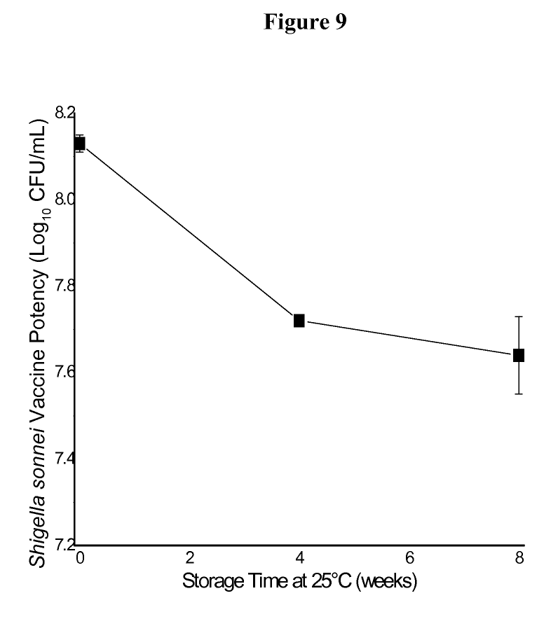

Ty21a-vectored Shigella sonnei vaccine, which is a Ty21a Salmonella strain stably expressing cloned genes controlling synthesis of the I O-polysaccharides (O-Ps) forms of Shigella sonnei (Xu, D. Q. et al (2002) Infect. Immun. 70, 4414-4423), was obtained from the laboratory of Dennis Kopecko (CBER, FDA). The strain was cultured by inoculation in BHI broth overnight and harvested in the stationary phase (2.2 OD_{600 nm}). The sample was centrifuged at 2500 rcf for 10 minutes, and the resulting bacterial pellet was resuspended in formulation 12 (Table 2) and taken to the initial volume. 0.5 mL aliquot of the bacterial sample was placed into each vial and dried according to cycle 1. The vials were sealed under slight vacuum (˜650 Torr) in argon gas, crimped, and stored at 25° C. The vials were taken out at various time points and then reconstituted with double-filtered deionized water. The samples were plated out on TSB plates for viability determination (FIG. 9). The plates were counted after 16 hours of incubation at 37° C.

### Example 11

### Stability of Cavitation-Dried Ty21a-Vectored Anthrax PA Vaccine at Room Temperature

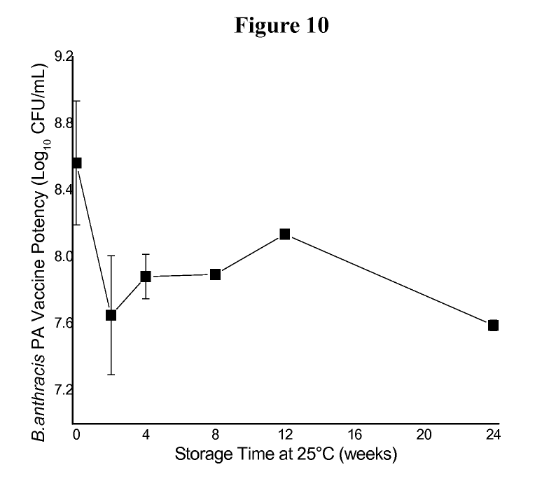

Ty21a-vectored anthrax protective antigen (PA) vaccine, which is an episomal pGB-2 plasmid regulated by the nirB promoter used to drive the expression of wild type B. anthracis PA gene (2295 bp), was obtained from the laboratory of Dennis Kopecko (CBER, FDA). The strain was cultured by inoculation in BHI broth overnight and harvested in the stationary phase (2.2 OD_{600 nm}). The sample was centrifuged at 2500 rcf for 10 minutes, and the resulting bacterial pellet was resuspended in formulation 9 (Table 2) and taken to the initial volume. 0.5 mL aliquot of the bacterial sample was placed into individual vials and dried according to cycle 1. The vials were sealed under slight vacuum (˜650 Torr) in argon gas, crimped, and stored at 25° C. The vials were taken out at various time points and then reconstituted with double-filtered deionized water. The samples were plated out on TSB plates for viability determination (FIG. 10). The plates were counted after 16 hours of incubation at 37° C.

### Example 12

### Stability of Freeze Dried Ty21a at Room Temperature

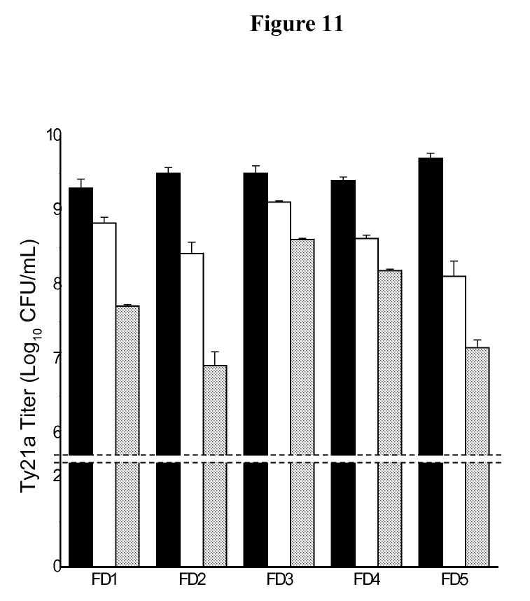

Ty21a was cultured by inoculation in BHI broth overnight and harvested in the stationary phase (2.2 OD_{600 nm}). The sample was centrifuged at 2500 rcf for 10 minutes, and the resulting bacterial pellet was resuspended in the formulations shown below in Table 3, and taken to the initial volume. 0.5 mL aliquot of the bacterial sample was placed into individual vials, pre-frozen prior to lyophilization by liquid N₂, (the bacteria were submerged in liquid nitrogen until frozen; typically, a 1 mL sample in a 10 cc vial was submerged for 30-60 seconds) and then freeze dried according to: primary drying conducted for 24 hours at −32° C. at 50 mTorr followed by secondary drying at 10° C. for 48 hours. Secondary drying was conducted while maintaining pressure at 50 mTorr. The vials were sealed under slight vacuum (˜650 Torr) in argon gas, crimped, and stored at 25° C. The vials were taken out after 1 week and then reconstituted with double-filtered deionized water. The samples were plated out on TSB plates for viability determination (FIG. 11). The plates were counted after 16 hours of incubation at 37° C.

TABLE 3
Formulation Compositions for Lyophilized Ty21a

| Components | FD1 | FD2 | FD3 | FD4 | FD5 |
|---|---|---|---|---|---|
| Sucrose (%, w/v) | 7 | 7 | 14 | 28 | 7 |
| Gelatin (%, w/v) | 1 | 5 | 1 | 1 | 1 |
| Methionine (%, w/v) | | | | | 1 |
| KPO₄ (mM) | 25 | 25 | 25 | 25 | 25 |
| pH | 7 | 7 | 7 | 7 | 7 |

### Example 13

### Recovery of Ty21a Following Spray Drying

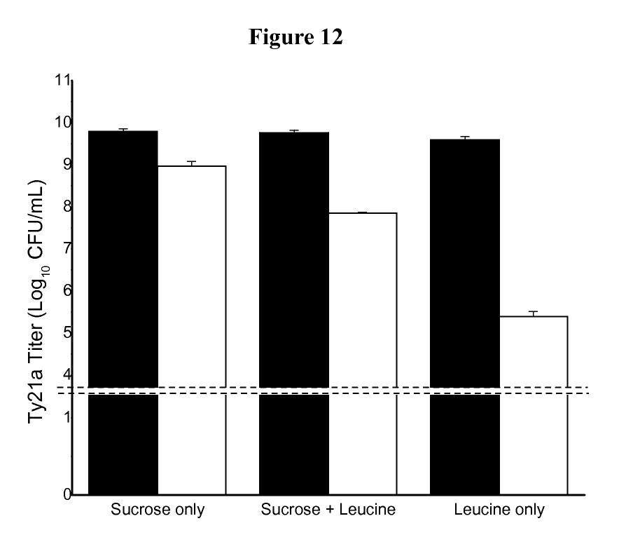

Ty21a was cultured by inoculation in BHI broth overnight and harvested in the stationary phase (2.2 OD_{600 nm}). The sample was centrifuged at 2500 rcf for 10 minutes, and the resulting bacterial pellet was resuspended in the formulations shown below in Table 4, and taken to the initial volume. The resulting solutions were spray dried using a Büchi 190 mini-spray dryer under the condition of: 0.75 mL/min solution feed rate, 45° C. inlet temperature, and 35° C. outlet temperature. The recovered powder was reconstituted with double-filtered deionized water and plated out on TSB plates for viability determination (FIG. 12). The plates were counted after 16 hours of incubation at 37° C.

TABLE 4
Formulation Compositions for Spray Dried Ty21a

| Components | SD1 | SD2 | SD3 |
|---|---|---|---|
| Sucrose (%, w/v) | 7 | | 7 |
| Leucine (%, w/v) | | 2 | 2 |

### Example 14

### Stability of Spray Dried Ty21a at Room Temperature

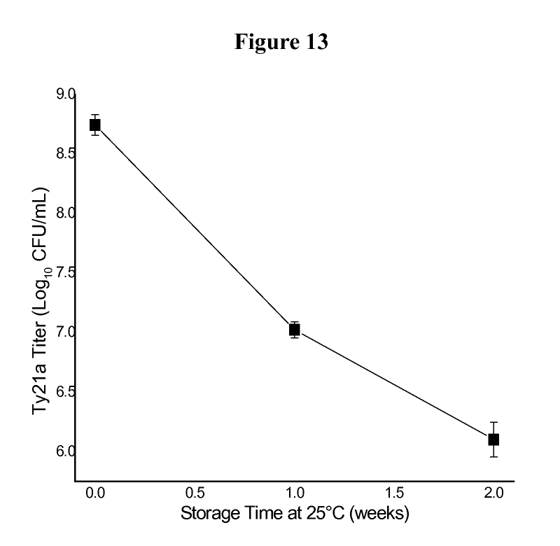

Live Ty21a was cultured by inoculation in BHI broth overnight and harvested in the stationary phase (2.2 OD_{600 nm}). The sample was centrifuged at 2500 rcf for 10 minutes, and the resulting bacterial pellet was resuspended in a formulation containing 3% (w/v) trehalose, 7% (w/v) sucrose, 0.02% (wt) Pluronic® F68, 0.25% (wt) glycerol, in 25 mM potassium phosphate buffer adjusted to pH7 and taken to the initial volume. The resulting solution was spray dried using a Büichi 190 mini-spray dryer under the condition of: 0.75 mL/min solution feed rate, 45° C. inlet temperature, and 35° C. outlet temperature. The recovered powder was placed into vials, under condition of controlled relative humidity and temperature (30° C. and less than 5% RH), and stored at 25° C. The vials were taken out at various time points and then reconstituted with double-filtered deionized water. The samples were plated out on TSB plates for viability determination (FIG. 13). The plates were counted after 16 hours of incubation at 37° C. Ty21a titer decreased by 0.72 Log₁₀ CFU following spray drying.

### Example 15

### Effect of Formulation Components on the Process Recovery of Cavitation-Dried Francisella tularensis

Francisella tularensis LVS was cultured by inoculation in Mueller Hinton broth supplemented with 10% glucose, 2.5% ferric pyrophosphate, and Isovitalex®. The bacterial suspension was shaken at 220 rpm and 37° C. overnight and was harvested in the stationary phase (0.85 OD_{600 nm}). The sample was centrifuged at 4000 rcf for 10 minutes, and the resulting bacterial pellet was resuspended in the formulations shown below in Table 5 and taken to twice the initial volume. 1 mL aliquot of the bacterial sample was placed into individual vials and dried according to cycle 2. The vials were sealed under slight vacuum (˜650 Torr) in argon gas, and then crimped. The dried samples were reconstituted with double-filtered deionized water and were plated out on Mueller Hinton Agar plates to determine the process-associated loss in bacterial titer (Table 5). The agar plates were supplemented with 10% glucose, 2.5% ferric pyrophosphate, Isovitalex®, and fetal bovine serum. The plates were counted after 48 hours of incubation at 37° C. and 5% CO₂.

TABLE 5
Formulation Compositions for Cavitation-Dried Francisella

| Components | Fr1 | Fr2 | Fr3 | Fr4 | Fr5 | Fr6 | Fr7 | Fr8 |
|---|---|---|---|---|---|---|---|---|
| Trehalose | 30 | 30 | 30 | 30 | 30 | 30 | 30 | 30 |
| (%, w/v) | | | | | | | | |
| Gelatin | | | | | | 5 | 5 | 5 |
| (%, w/v) | | | | | | | | |
| Pluronic F68 | | | | | | | | 0.02 |
| (wt %) | | | | | | | | |
| Methionine | | 0.5 | | | | 2 | | |
| (%, w/v) | | | | | | | | |
| Arginine | | | | 0.5 | | | | |
| (%, w/v) | | | | | | | | |
| Glutamate | | | | | 0.5 | | | |
| (%, w/v) | | | | | | | | |
| DMSO (wt %) | | | 0.5 | | | | 0.5 | |
| KPO₄ (mM) | 25 | 25 | 25 | 25 | 25 | 25 | 25 | 25 |
| pH | 8 | 8 | 8 | 8 | 8 | 8 | 8 | 8 |
| Process Loss | 0.79 | 0.57 | 0.87 | 0.26 | 0.39 | 0.41 | 0.74 | 0.96 |
| (Log₁₀) | | | | | | | | |

### Example 16

### Effect of Formulation Components on the Storage Stability of Cavitation-Dried Francisella tularensis

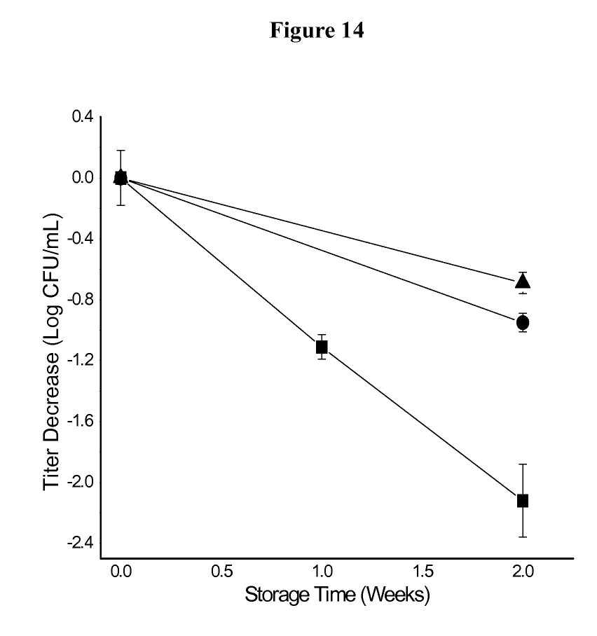

Francisella tularensis LVS was cultured by inoculation in Mueller Hinton broth supplemented with 10% glucose, 2.5% ferric pyrophosphate, and Isovitalex®. The bacterial suspension was shaken at 220 rpm and 37° C. overnight and was harvested in the stationary phase (0.85 OD_{600 nm}). The sample was centrifuged at 4000 rcf for 10 minutes, and the resulting bacterial pellet was resuspended in formulation Fr6, shown in Table 5, and taken to twice the initial volume. 1 mL aliquot of the bacterial sample was placed into individual vials and dried according to cycle 2. The vials were sealed under slight vacuum (˜650 Torr) in argon gas, crimped, and stored at various temperatures including 4, 25, 37, and 45° C. The dried samples were reconstituted with double-filtered deionized water and were plated out on Mueller Hinton Agar plates to determine the process-associated loss and storage stability-associated loss in bacterial titer (FIG. 14). The agar plates were supplemented with 10% glucose, 2.5% ferric pyrophosphate, Isovitalex®, and fetal bovine serum. The plates were counted after 48 hours of incubation at 37° C. and 5% CO₂.

### Example 17

### Effect of Formulation Components on the Storage Stability of Cavitation-Dried Francisella tularensis

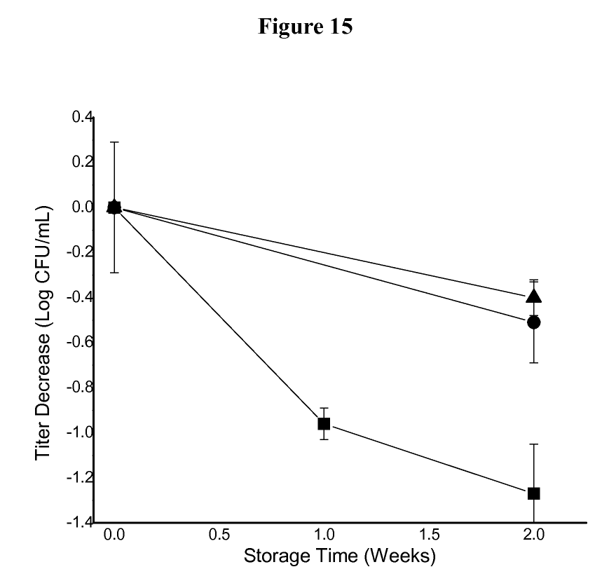

Francisella tularensis LVS was cultured by inoculation in Mueller Hinton broth supplemented with 10% glucose, 2.5% ferric pyrophosphate, and Isovitalex®. The bacterial suspension was shaken at 220 rpm and 37° C. overnight and was harvested in the stationary phase (0.85 OD_{600 nm}). The sample was centrifuged at 4000 rcf for 10 minutes, and the resulting bacterial pellet was resuspended in 30% (w/v) trehalose, 5% (w/v) gelatin, and 25 mM KPO₄ (pH8) and taken to twice the initial volume. lmL aliquot of the bacterial sample was placed into individual vials and dried according to cycle 2. The vials were sealed under slight vacuum (˜650 Torr) in argon gas, crimped, and stored at various temperatures including 4, 25, 37, and 45° C. The dried samples were reconstituted with double-filtered deionized water and were plated out on Mueller Hinton Agar plates to determine the process-associated loss and storage stability-associated loss in bacterial titer (FIG. 15). The agar plates were supplemented with 10% glucose, 2.5% ferric pyrophosphate, Isovitalex®, and fetal bovine serum. The plates were counted after 48 hours of incubation at 37° C. and 5% CO₂.

### Example 18

### Effect of Formulation Components on the Process Recovery of Dried Measles Virus

Edmonton-Zagreb live attenuated measles virus vaccine was grown in Vero cells to a titer of approximately 6.0 log₁₀ (TCID₅₀). The virus was formulated in 8.3% (w/v) trehalose, 12.7% (w/v) sucrose, 4% (w/v) L-arginine, 1.25 wt % glycerol, 0.06 wt % Pluronic F68, and 50 mM KPO₄ adjusted to pH7. The virus titer was adjusted to 4.0 log₁₀. 10 mL vials with a fill volume of 1 mL were used. The samples were placed on the freeze dryer shelf at 15° C. and allowed to equilibrate for 10 minutes and dried according to cycle 1. The vials were sealed under slight vacuum (˜650 Torr) in argon gas, crimped, and stored at 37° C. The dried samples were reconstituted with double-filtered deionized water and their viabilities were determined using the tissue culture infectious dose (TCID₅₀) assay (Table 6).

TABLE 6
Storage Stability of Cavitation-Dried Measles Virus at 37° C.

| Starting Material | Post-process | 1 week | 2 week |
|---|---|---|---|
| 4.0 | 3.0 ± 0.57 | 2.6 ± 0.24 | 2.3 ± 0.00 |

### Example 19

### Stabilization by Methionine

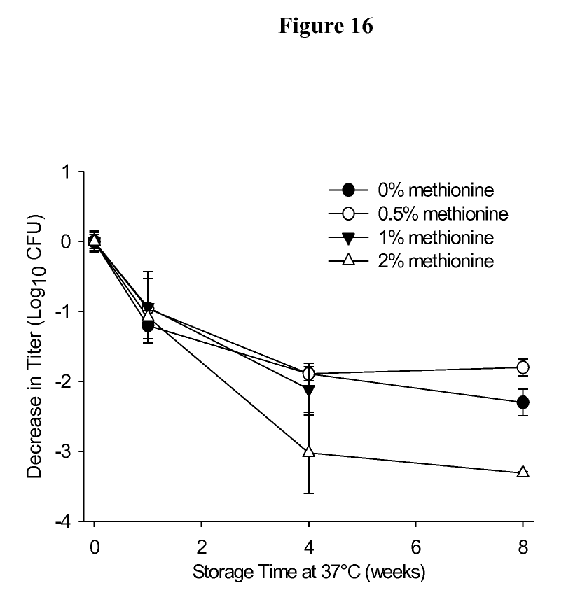

Methionine was added to the trehalose-potassium phosphate formulation at pH8, ranging in concentration from 0.5-2% (w/v). Incorporation of 0.5% (w/v) methionine improved storage stability, as the titer loss upon storage was reduced from 2.1 Log₁₀ to 1.8 Log₁₀, following 8 weeks of storage at 37° C. At higher methionine concentrations (i.e., 2%), however, the stability worsened, resulting in titer loss greater than 3 Log₁₀ (FIG. 16). The foam drying process-associated decrease in titer was also higher for formulations containing higher methionine concentrations, 0.92 Log₁₀ compared to 0.45 Log₁₀ loss for formulations containing 2 and 0.5% (w/v) methionine, respectively (data not shown). Thus, optimal storage stability was observed for cavitation-dried Ty21a in the presence of 0.5% methionine.

### Example 20

### Stabilization by Plasticizers

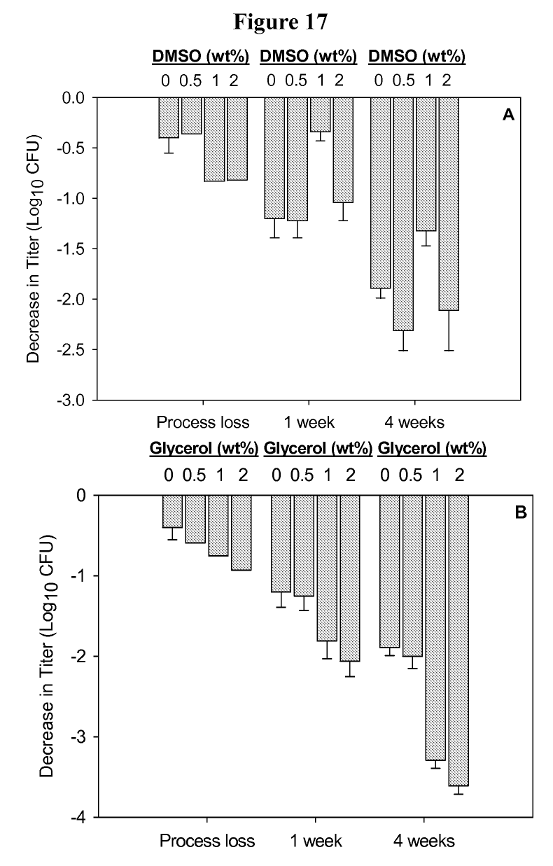

The addition of plasticizers has been demonstrated to improve the storage stability of various proteins and enzymes when incorporated to a sugar solution upon dehydration. DMSO and glycerol, ranging in concentration from 0.5 to 2 wt %, were incorporated into the trehalose-potassium phosphate buffered formulation at pH8 and then foam dried. The foam drying process loss ranged from 0.3 to 1.0 Log₁₀, with the higher loss associated with higher plasticizer concentrations (FIG. 17A,B). The storage stability of the vaccine was significantly improved with the inclusion of DMSO. Formulation containing DMSO at 1 wt % reduced the stability loss to 1.3 Log₁₀ following 4 weeks of storage at 37° C., while all other compositions resulted in >2 Log₁₀ decrease (FIG. 17A). In the absence of plasticizers, the stability loss was 1.9 Log₁₀. In comparison to DMSO, glycerol was not as effective a stabilizer; Ty21a containing 0.5 wt % glycerol demonstrated similar stability to the formulation without any plasticizers, while the inclusion of higher concentrations of glycerol resulted in decreased stability (FIG. 17B). The addition of DMSO typically resulted in improved storage stability compared to foam dried Ty21a formulated with equal concentrations of glycerol (see FIG. 17A,B for comparison at 1 wt % inclusion). Optimal storage stability was obtained with cavitation-dried Ty21a containing 1 wt % DMSO.

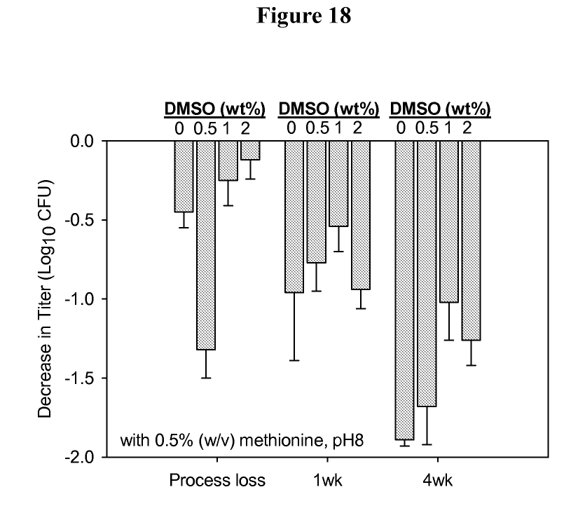

The addition of methionine (FIG. 16) and DMSO (FIG. 17A), individually, was shown to have a stabilizing effect on foam dried Ty21a. The effect of both components on the storage stability of Ty21a was evaluated next. While maintaining the methionine composition at 0.5% (w/v), the DMSO concentration was varied from 0 to 2 wt % (FIG. 18). All formulations, with the exception of that prepared with 0.5wt % DMSO, demonstrated less than 0.5 Log₁₀ loss upon foam drying. The optimal amount of DMSO, 1 wt %, was the same as that observed previously in the absence of methionine (FIG. 17A). An additive effect on the storage stability of Ty21a was observed upon the inclusion of both components to the trehalose formulation. While Ty21a formulated in 0.5% (w/v) methionine decreased in titer by 1.9 Log₁₀ (FIG. 16) and that in 1 wt % DMSO by 1.3 Log₁₀ (FIG. 17A), the decrease in titer for Ty21a containing both methionine and DMSO was minimized to 1 Log₁₀ (FIG. 18). In other words, the minimal decrease in titer of 1 Log₁₀, found in the formulation containing both methionine and DMSO, constituted a smaller decrease than that found with methionine only, or with DMSO only. Similar additive effect was also observed for the formulation prepared at pH7 (data not shown).

### Example 21

### Stabilization by Gelatin

Gelatin was incorporated into the formulation to not only enhance the vaccine stability but also enhance solution viscosity, which can minimize the rate of cavitation associated with the foaming process. The effect of gelatin addition on the storage stability of foam dried Ty21a was examined on formulations containing trehalose along with another excipient selected from methionine, glycerol, or DMSO. The storage stability data for the same samples lacking gelatin were presented earlier and their slopes of titer decrease are shown in Table 7. Upon the inclusion of gelatin, the storage stability of foam dried Ty21a was improved, irrespective of the formulation composition; for the formulation containing methionine and trehalose, gelatin addition reduced the slope of titer decrease from −0.2 Log₁₀/wk to −0.12 Log₁₀/wk, for glycerol and trehalose from −0.36 Log₁₀/wk to −0.18 Log₁₀/wk, and for DMSO and trehalose from −0.25 Log₁₀/wk to −0.18 Log₁₀/wk (Table 7). From these combinations, a formulation containing methionine with gelatin and trehlaose was observed to be the most stable formulation for foam dried Ty21a, demonstrating over 8 weeks of storage stability at 37° C. (time to 1 Log₁₀ loss).

Stability of foam dried Ty21a in various formulations stored at 37° C. (Table 7). The effects of gelatin incorporation are examined in the presence of three other excipients, methionine, glycerol, and DMSO. In all of the formulations examined, gelatin addition improved the storage stability of foam dried Ty21a.

TABLE 7

Slope (Log₁₀/wk) at 37° C.

| | Methionine³ | Glycerol⁴ | DMSO⁵ |
|---|---|---|---|
| Without gelatin¹ | −0.20 ± 0.04 | −0.36 ± 0.00 | −0.25 ± 0.00 |
| With gelatin² | −0.12 ± 0.03 | −0.18 ± 0.02 | −0.18 ± 0.04 |

¹Formulation further contained 30% (w/v) trehalose and 25 mM KPO₄ at pH 8
²Formulation further contained 25% (w/v) trehalose, 5% (w/v) gelatin, and 25 mM KPO₄ at pH 8
³Methionine present at 1% (w/v)
⁴Glycerol present at 1 wt %
⁵DMSO present at 1 wt %

### Example 22

### Effect of Formulation Components on the Process Recovery of Dried Adjuvanted Vaccine

Recombinant B. anthracis with 80 kilodalton recombinant antigen was formulated in the presence of alum-adjuvant and oligonucleotide. 0.2 mg/mL dmPA was initially mixed with 1.5 mg/mL alum (Alhydrogel®, Al(OH)₃) for 30 min, followed by the addition of 1.0 mg/mL oligonucleotide, and the formulation components were added, resulting in the formulation composition shown in Table 8. 10 mL vials with a fill volume of 1 mL were used. The samples were placed on the freeze dryer shelf at 15° C. and allowed to equilibrate for 10 minutes and dried according to cycle 2. The vials were sealed under slight vacuum (˜650 Torr) in argon gas and then crimped. The dried samples were reconstituted with double-filtered deionized water and the remaining activities were determined using a cell-based assay to assess the inhibitory capability of the cavitation-dried, adjuvanted vaccine against a recombinant lethal factor (Table 8).

TABLE 8

| Components¹ | 1 | 2 | 3 | 4 | 5 | 6 | 7 | 8 | 9 | 10 | 11 | 12 | 13 | 14 | 15 |
|---|---|---|---|---|---|---|---|---|---|---|---|---|---|---|---|
| Trehalose | | | | 30 | 29.4 | | | | 27 | 26.4 | | | | 29.4 | 30 |
| Sucrose | 30 | 29.4 | 29.4 | | | 30 | 29.1 | 30 | | | 27 | 26.4 | 30 | | |
| Glycerol | | 0.6 | | | | | | | | | | | | | |
| Sorbitol | | | 0.6 | | 0.6 | | | | | 0.6 | | 0.6 | | 0.6 | |
| Polysorbate 80 | | | | | | | | 0.005 | | | | | | | |
| Arginine | | | | | | | | | 3 | 3 | 3 | | | | |
| NaCl | | | | | | | 0.9 | | | | | | | | |
| NaPO₄ (mM) | | | | | | | | | | | | | 5 | 5 | 5 |
| Tris (mM) | 20 | 20 | 20 | 20 | 20 | 20 | 20 | 20 | 20 | 20 | 20 | 20 | | | |
| pH | 7.4 | 7.0 | 7.4 | 7.0 | 7.4 | 7.4 | 7.4 | 7.4 | 7.4 | 7.4 | 7.4 | 7.4 | 7.4 | 7.4 | 7.4 |
| Process Loss (%)² | 27 | 22 | 9 | 0 | 0 | 24 | 14 | 0 | 18 | 10 | 7 | 12 | 46 | 30 | 0 |

¹Component composition given as % (w/v), unless stated otherwise.
²Calculated process loss based on pre- and post-cavitation dried activity, as determined by the cell-based assay examining the activity of dmPA in inhibiting a lethal factor.

### Example 23

Details of the cavitation drying procedure of cycle 2 were carried out as follows. These procedures were carried out using a lyophilizer containing 60 mL of 40% sucrose solution. The time and temperature for each step is identified. The transition time in going from one step to the next step is also identified. Details from two separate runs are shown below. Both runs #1 and #2 are “ramp-down” in the sense that the procedures were stopped at intermediate pressures, that is, 10, 4, and 1 Torr. The lyophilizer was, Virtis Advantage 2.0. Maximum Condenser Capacity=3.5 L

TABLE 9
Steps used in drying procedure, with 60 mL of 40% sucrose solution,
where this procedure is well-suited for cavitation-drying.

| Run #1 with 60 mLs in | Run #2 with 60 mLs in |
|---|---|
| 6 beakers. | 60 vials. |
| Pressure drop from | Pressure drop from |
| 760 to 10 Torr. | 760 to 10 Torr. |
| Time: 2 min 38 sec | Time: 2 min 52 sec |
| Pressure held at 10 Torr. | Pressure held at 10 Torr. |
| Time: 10 min | Time: 10 min |
| Pressure drop from | Pressure drop from |
| 10 to 4 Torr. | 10 to 4 Torr. |
| Time: 37 sec | Time: 26 sec |
| Pressure held at 4 Torr. | Pressure held at 4 Torr. |
| Time 10 min | Time 10 min |
| Pressure drop from | Pressure drop from |
| 4 to 1 Torr. | 4 to 1 Torr. |
| Time: 21 sec | Time: 25 sec |
| Pressure held at 1 Torr. | Pressure held at 1 Torr. |
| Time: 15 min | Time: 15min |
| Pressure drop from | Pressure drop from |
| 1 to 0.10 Torr. | 1 to 0.10 Torr. |
| Time: 1 min 14 sec | Time: 1 min 20 sec |

TABLE 10
Runs #4 and #5 resemble cycle 2,
except the final pressure is 1 Torr.

#1: Virtis Advantage 2.0 with the 3.5 L Condenser Capacity
(the 3.5 L unit has an integrated vacuum control):
What: 60 mL of 40% SC Sucrose
Cycle: 760 --> 0.1 Torr
Time to ramp down: 7 m 16 sec
#2: Virtis Advantage 2.0 with the 3.5 L Condenser Capacity:
What: 60 mL of 40% SC Sucrose
Cycle: 760 --> 1 Torr
Time to ramp down: 3 m 38 sec
#3: Virtis Advantage 2.0 with a 25 L Condenser Capacity
(Freezemobile 25EL condenser.
Thyracount Vacuum Controller DC1S was added
to control the pressure in the chamber):
What: 60 mL of 40% SC Sucrose
Cycle: 760 --> 1 Torr
Time to ramp down: 3 m 32 sec
#4: Virtis Advantage 2.0 with a 25 L Condenser Capacity
(Freezemobile 25EL condenser.
Thyracount Vacuum Controller DC1S was added
to control the pressure in the chamber):
What: 60 mL of 40% SC Sucrose
Cycle: 760 --> 10 --> 4 --> 1
Times of 3 ramp downs respectively: 2 m 13 sec, 36 sec, 50 sec.
Holds at 10 Torr and 4 Torr: 10 min
#5: Virtis Advantage 2.0 with a 25 L Condenser Capacity
(Freezemobile 25EL condenser.
Thyracount Vacuum Controller DC1S was added
to control the pressure in the chamber):
What: Air
Cycle: 760 --> 10 --> 4 --> 1
Times of 3 ramp downs respectively: 2 m 05 sec, 33 sec, 1 min.
Holds at 10 Torr and 4 Torr: 10 min

Although the foregoing invention has been described by way of descriptions, data, and examples, it will be apparent to those skilled in the art that various changes and modifications can be practiced without departing from the spirit of the invention. Therefore the foregoing descriptions, data, and examples should not be construed as limiting the scope of the invention. All patents and published patent applications, identified herein, are incorporated by reference.

What is claimed is:

1. A cavitation-dried composition that comprises a biologically active sample,
wherein the composition is prepared from a mixture of the biologically active sample and a formulation,
wherein the mixture contains
a polyol (20-70% w/v of mixture), and
a plasticizer (0.1-10.0% w/w of mixture),
at pH 6.0-8.5,
wherein to prepare the composition, the mixture is subjected to a vacuum,
wherein the mixture temperature, or shelf temperature, is maintained above the freezing point of the mixture,
wherein a cavitation-dried structure is produced, and wherein the cavitation-dried structure is a foam or a film.

2. The cavitation-dried composition of claim 1, wherein the polyol is sucrose (20-50% w/v in mixture), trehalose (20-50% w/v in mixture), or combinations thereof.

3. The cavitation-dried composition of claim 1, wherein the plasticizer is glycerol, dimethylsulfoxide (DMSO), propylene glycol, ethylene glycol, oligomeric polyethylene glycol, or sorbitol.

4. The cavitation-dried composition of claim 1, wherein the formulation contains an amino acid, where the concentration of the amino acid in the mixture is 0.5-5.0% w/v.

5. The cavitation-dried composition of claim 1, wherein the formulation contains an amino acid, and wherein the amino acid is methionine, arginine, or combinations thereof, wherein the amino acid in the mixture is 0.5-5.0% w/v.

6. The cavitation-dried composition of claim 1, that contains a surfactant, wherein the concentration of the surfactant in the mixture is 0.01-5.0% w/v.

7. The cavitation-dried composition of claim 1, that contains a surfactant, and is a surfactant-containing composition, and
wherein there is a ratio of residence of the biologically active sample at the surface of the surfactant-containing composition versus at the interior of the surfactant-containing composition,
and wherein the surfactant results in a decrease in this ratio,
as compared to the ratio in a second cavitation-dried composition that contains all of the components of the surfactant-containing composition but lacking the surfactant.

8. The cavitation-dried composition of claim 1, that further comprises a surfactant, wherein the surfactant has the chemical composition of Tween20®, Span20®, Tween80®, or Pluronic® poloxamer.

9. The cavitation-dried composition of claim 1, that has a specific surface area, and where the specific surface area is less than 0.3 meters squared per gram of mass.

10. The cavitation-dried composition of claim 1, that has a specific surface area, and where the specific surface area is less than 0.1 meters squared per gram of mass.

11. The cavitation-dried composition of claim 1 that is a film.

12. The cavitation-dried composition of claim 1, wherein the formulation contains
an amino acid and a plasticizer,
wherein the amino acid reduces process loss,
wherein the plasticizer reduces process loss, or
wherein the combination of both amino acid and plasticizer in the formulation has an additive effect in reducing process loss.

13. The cavitation-dried composition of claim 12, wherein the amino acid is methionine and the plasticizer is dimethylsulfoxide (DMSO)

14. The cavitation-dried composition of claim 1, wherein the formulation is from Table 1, 2, 5, or 8.

15. The cavitation-dried composition of claim 1, wherein the biologically active sample is bacteria or viruses.

16. The cavitation-dried composition of claim 1, wherein the biologically active sample is bacteria, and wherein
(a) the bacteria is harvested at stationary phase,
(b) the bacterial growth medium is hyperosmotic, or
(c) the bacteria is harvested at stationary phase and the bacterial growth medium is hyperosmotic,
wherein process stability is increased in the hyperosmotic medium compared to process stability wherein the bacterial growth medium is iso-osmotic, and wherein process stability is increased when the bacteria are harvested in stationary phase compared to process stability when the bacteria are harvested in the log phase.

17. The cavitation-dried composition of claim 1, wherein the biologically active sample does not elicit an immune response against itself, or is engineered to prevent an immune response against itself.

18. The cavitation-dried composition of claim 1, wherein the biologically active sample can elicit an immune response against itself.

19. A method for preparing a cavitation-dried composition of a biologically active sample, from a mixture of a biologically active sample and a formulation,
wherein the formulation comprises a polyol, and
a plasticizer or a surfactant,
and wherein the mixture is in a container, wherein the method comprises
decreasing the chamber pressure in a stepwise manner to reduce the water content of the mixture,
wherein the mixture temperature or the shelf temperature, is maintained above the freezing point of the mixture,
wherein a foam or film is produced, and
wherein the mixture does not freeze and the foam or film does not freeze.

20. The method of claim 19, wherein the longest step of the stepwise manner takes at least 3 minutes.

21. The method of claim 19, wherein the longest step of the stepwise manner takes at least 10 minutes.

22. The method of claim 19, wherein there is a transition time in between two consecutive pressures, and wherein the transition time is selected from a time that is at least 1, 2, 10, 20, 60, and 120 minutes.

23. The method of claim 19, wherein each step of the stepwise manner takes at least 3 minutes or at least 10 minutes

24. The method of claim 19, wherein the stepwise manner contains at least two steps.

25. The method of claim 19, wherein the stepwise manner contains at least three steps.

26. The method of claim 19, wherein the biologically active sample is a bacteria, a virus, a protein, an adjuvanted protein, or is a pharmaceutical antibody.

27. The method of claim 19, wherein the mixture temperature, or shelf temperature is maintained at or above about 10 degrees C.

28. The method of claim 19, wherein the mixture temperature, or shelf temperature, is maintained at 15-25 degrees C.

29. The method of claim 19, wherein the biologically active sample is bacteria, and
wherein the bacteria used for the method are harvested in the stationary phase, and then used to form the mixture of the formulation and bacteria,
and wherein the process stability of the cavitation-dried composition of bacteria is increased,
where the increase in process stability is relative to that of a cavitation-dried composition, where bacteria are harvested in the log phase.

30. The method of claim 19, wherein the biologically active sample is bacteria, and wherein the bacteria used for the method are prepared by growing in hyperosmotic growth medium, and then used to form the mixture of the formulation and bacteria,
and wherein the process stability of the cavitation-dried composition of bacteria is increased,
where the increase in process stability is relative to that of a cavitation-dried composition where bacteria are prepared by growing in an iso-osmotic growth medium.

31. The method of claim 30, wherein the hyperosmotic growth medium contains 0.2-1.0 M NaCl.

32. The method of claim 19, wherein the pressure is decreased in a stepwise manner from about 10 Torr to less than about 100 mTorr.

33. The method of claim 19, wherein there is a primary drying pressure, wherein the primary drying pressure used in the cavitation drying process is reached within about three hours.

34. The method of claim 19, wherein the polyol is 20% to 70% w/v of the mixture, and wherein the mixture contains a plasticizer that is 0.1% to 10.0% w/v of the mixture.

35. The method of claim 19, wherein the stability of the cavitation-dried composition is increased, relative to the stability of a cavitation-dried composition where the mixture is made by combining the biological sample with a formulation that does not contain a polyol.

36. The method of claim 19, wherein the cavitation-dried composition contains a polymer, and
is a polymer-containing composition,
wherein the stability of the polymer-containing composition is increased, relative to
a composition that contains all of the components of the polymer-containing composition
but does not contain the polymer.

37. The method of claim 33, wherein the polymer is one or more of gelatin, partially hydrolyzed gelatin, collagen, chondroitin sulfate, sialated polysaccharide, polyvinyl pyrrolidone, actin, myosin, microtubule protein, or serum albumin.

38. The method of claim 19, wherein the formulation comprises 20-50% trehalose and 0-10% gelatin at pH 7-8.

39. The method of claim 19, wherein the polyol is one or more of sucrose or trehalose.

40. The method of claim 19, wherein the formulation contains methionine, and
wherein the methionine content of the mixture is about 0.5%,
wherein the cavitation-dried composition is a methionine-containing composition, and
wherein the storage stability of the methionine-containing composition is increased,
relative to the storage stability of a cavitation-dried composition produced having the same components of the mixture used to make the methionine-containing composition,
except that the formulation does not contain methionine.

41. The method of claim 19, wherein the formulation contains a plasticizer, and
wherein the cavitation-dried composition is a plasticizer-containing composition, and
wherein the storage stability of the plasticizer-containing composition is increased,
relative to the storage stability of a cavitation-dried composition produced having the same components of the mixture used to make the plasticizer-containing composition,
except that the formulation does not contain plasticizer.

42. The method of claim 19, wherein the concentration of the plasticizer in the formulation is
dimethylsulfoxide (DMSO), where the DMSO content of the mixture is about 0.5%-2.0% w/v,
or glycerol, where the glycerol content of the mixture is about 0.5%-2.0% w/v.

43. The method of claim 19, wherein the formulation contains gelatin (0.1-10%), and wherein the composition is a gelatin-containing composition,
wherein the storage stability of the gelatin-containing composition is increased,
relative to the storage stability of a cavitation-dried composition produced having the same components of the mixture used to make the gelatin-containing composition,
except that the formulation does not contain gelatin.

44. The method of claim 19, wherein the biologically active sample is Salmonella, Shigella, Listeria, Franciscella, Escherichia coli, Pneumococcus, Mycobacterium, Pseudomonas, Staphylococcus, Streptococcus, or Bacillus anthracis.

45. The method of claim 19, where the method additionally comprises the step of mixing the formulation with the biologically active sample to form the mixture.

46. The method of claim 45, wherein the method additionally comprises the step of harvesting the bacteria in the stationary phase.

47. The method of claim 45, wherein the method additionally comprises the step of growing the bacteria in hyperosmotic growth medium.

48. The method of claim 19, wherein the cavitation-dried composition has a specific surface area, and where the specific surface area is less than 0.3 meters squared per gram of mass.

49. The method of claim 19, wherein the cavitation-dried composition has a specific surface area, and where the specific surface area is less than 0.1 meters squared per gram of mass.

50. The method of claim 19, wherein the cavitation-dried composition is a film.

51. The method of claim 19, wherein the cavitation-dried composition contains a surfactant, and is a surfactant-containing composition, and
wherein there is a ratio of residence of the biologically active sample at the surface of the surfactant-containing composition versus at the interior of the surfactant-containing composition,
and wherein the surfactant results in a decrease in this ratio,
as compared to the ratio in a second cavitation-dried composition that contains all of the components of the surfactant-containing composition but lacking the surfactant.

52. A cavitation-dried composition of a biological sample prepared according to the method of claim 19.

53. A formulation for preparing a cavitation-dried composition that comprises a biologically active sample, wherein the formulation contains
a polyol (20-70% w/v), and
a plasticizer (0.1-10.0% w/w),
at pH 6.0-8.5.

54. The formulation of claim 53, that is selected from a formulation of Tables 1, 2, 5, and 8.
